(19) United States
(12) Patent Application Publication
(10) Pub. No.: US 2006/0183901 A1
(43) Pub. Date: Aug. 17, 2006

(54) Crystalline form of an indazole-carboxamide compound

(75) Inventors: Paul R. Fatheree, San Francisco, CA (US); S. Derek Turner, San Francisco, CA (US); Robert S. Chao, Santa Clara, CA (US); Jyanwei Liu, Sunnyvale, CA (US); Hao Zhang, Foster City, CA (US); Daniel Genov, San Francisco, CA (US); Junning Lee, El Granada, CA (US)

Correspondence Address: THERAVANCE, INC., 901 GATEWAY BOULEVARD, SOUTH SAN FRANCISCO, CA, 94080, US

(73) Assignee: THERAVANCE, INC.

(21) Appl. No.: 11/355,323

(22) Filed: Feb. 16, 2006

Related U.S. Application Data
(60) Provisional application No. 60/653,677, filed on Feb. 17, 2005.

(51) Int. Cl. C07D 403/14

(52) U.S. Cl. 544/363

(57) ABSTRACT

The invention provides crystalline halide salts of 1-isopropyl-1H-indazole-3-carboxylic acid {(1S,3R,5R)-8-[2-(4-acetylpiperazin-1-yl)ethyl]-8-azabicyclo[3.2.1]oct-3-yl}amide and solvates thereof. The invention also provides pharmaceutical compositions comprising such salts, methods of using such crystalline salts to treat diseases associated with 5-HT₄ receptor activity, and processes useful for preparing such crystalline salts.

### CROSS-REFERENCE TO RELATED APPLICATIONS

This application claims the benefit of U.S. Provisional Application No. 60/653,677 filed on Feb. 17, 2005, the disclosure of which is incorporated herein by reference in its entirety.

### BACKGROUND OF THE INVENTION

1. Field of the Invention

The invention is directed to crystalline salt forms of an indazole-carboxamide compound which are useful as 5-HT₄ receptor agonists. The invention is also directed to pharmaceutical compositions comprising such crystalline compounds, methods of using such compounds for treating medical conditions mediated by 5-HT₄ receptor activity, and processes useful for preparing such compounds.

2. State of the Art

Commonly-assigned U.S. Provisional Application No. 60/545,702, filed on Feb. 18, 2004, and U.S. patent application Ser. No. 11/060,195, filed on Feb. 17, 2005, disclose novel indazole-carboxamide compounds that are expected to be useful for the treatment of disorders of reduced motility of the gastrointestinal tract. In particular, the compound 1-isopropyl-1H-indazole-3-carboxylic acid {(1S,3R,5R)-8-[2-(4-acetylpiperazin-1-yl)ethyl]-8-azabicyclo[3.2.1]oct-3-yl}amide is specifically disclosed in these applications as demonstrating 5-HT₄ agonist activity.

The chemical structure of 1-isopropyl-1H-indazole-3-carboxylic acid {(1S,3R,5R)-8-[2-(4-acetylpiperazin-1-yl)ethyl]-8-azabicyclo[3.2.1]oct-3-yl}amide is represented by formula I:

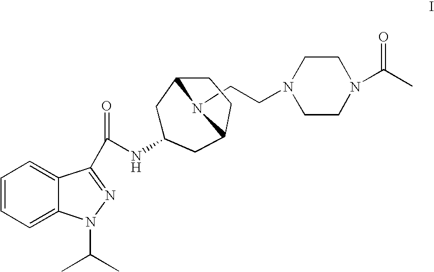

To effectively use this compound as a therapeutic agent, it would be desirable to have a solid-state salt form that can be readily manufactured and that has acceptable chemical and physical stability. For example, it would be highly desirable to have a salt form that is thermally stable, for example up to temperatures of about 150° C., and is not particularly hygroscopic nor deliquescent, thereby facilitating processing and storage of the material. Crystalline solids are generally preferred over amorphous forms, for enhancing purity and stability of the manufactured product.

No crystalline salt forms of the compound of formula I have previously been reported. Accordingly, a need exists for a stable, crystalline salt form of the compound of formula I that has an acceptable level of hygroscopicity, deliquescence, and thermal stability.

### SUMMARY OF THE INVENTION

The present invention provides crystalline halide salts of 1-isopropyl-1H-indazole-3-carboxylic acid {(1S,3R,5R)-8-[2-(4-acetylpiperazin-1-yl)ethyl]-8-azabicyclo[3.2.1]oct-3-yl}amide or a solvate thereof. In one aspect, the crystalline halide salt of the invention is a dihydrochloride salt of the compound of formula I. In another aspect, the crystalline halide salt is a dihydrobromide salt of the compound of formula I. In yet another specific aspect, the crystalline salt of the invention is a crystalline hydrate of the dihydrochloride salt of the compound of formula I.

Surprisingly, the crystalline halide salts of the invention have been found to be thermally stable at temperatures greater than about 150° C. and to exhibit weight changes of less than about 1% when exposed to a range of relative humidity between about 40% and about 60% at room temperature. Furthermore, the crystalline halide salts of the invention are not deliquescent when exposed to up to 60% relative humidity at room temperature.

Among other uses, the crystalline halide salts of a compound of formula I are expected to be useful for preparing pharmaceutical compositions for treating disorders of reduced motility of the gastrointestinal tract. Accordingly, in another of its composition aspects, the invention provides a pharmaceutical composition comprising a pharmaceutically-acceptable carrier and a crystalline halide salt of 1-isopropyl-1H-indazole-3-carboxylic acid {(1S,3R,5R)-8-[2-(4-acetylpiperazin-1-yl)ethyl]-8-azabicyclo[3.2.1]oct-3-yl}amide or a solvate thereof.

The invention also provides a method of treating a disease or condition associated with 5-HT₄ receptor activity, e.g. a disorder of reduced motility of the gastrointestinal tract, the method comprising administering to the mammal, a therapeutically effective amount of a a crystalline halide salt of 1-isopropyl-1H-indazole-3-carboxylic acid {(1S,3R,5R)-8-[2-(4-acetylpiperazin-1-yl)ethyl]-8-azabicyclo[3.2.1]oct-3-yl}amide or a solvate thereof.

In another method aspect, the invention provides a process for preparing a crystalline halide salt of the invention, the process comprising contacting 1-isopropyl-1H-indazole-3-carboxylic acid {(1S,3R,5R)-8-[2-(4-acetylpiperazin-1-yl)ethyl]-8-azabicyclo[3.2.1]oct-3-yl}amide with a halide acid.

The invention also provides a crystalline halide salt of the invention as described herein for use in therapy or as a medicament, as well as the use of a crystalline halide salt of the invention in the manufacture of a medicament, especially for the manufacture of a medicament for treating a disorder of reduced motility of the gastrointestinal tract in a mammal.

### BRIEF DESCRIPTION OF THE DRAWINGS

Various aspects of the present invention are illustrated by reference to the accompanying drawings.

FIG. 1 shows a powder x-ray diffraction (PXRD) pattern of a crystalline 1-isopropyl-1H-indazole-3-carboxylic acid {(1S,3R,5R)-8-[2-(4-acetylpiperazin-1-yl)ethyl]-8-azabicyclo[3.2.1]oct-3-yl}amide dihydrochloride salt of the invention.

FIG. 2 shows a differential scanning calorimetry (DSC) trace (bottom trace at left side of figure, left side vertical axis) and a thermal gravimetric analysis (TGA) trace (top trace, right side vertical axis) for a crystalline 1-isopropyl-1H-indazole-3-carboxylic acid {(1S,3R,5R)-8-[2-(4-acetylpiperazin-1-yl)ethyl]-8-azabicyclo[3.2.1]oct-3-yl}amide dihydrochloride salt of the invention.

FIG. 3 shows a powder x-ray diffraction (PXRD) pattern of a crystalline 1-isopropyl-1H-indazole-3-carboxylic acid {(1S,3R,5R)-8-[2-(4-acetylpiperazin-1-yl)ethyl]-8-azabicyclo[3.2.1]oct-3-yl}amide dihydrobromide salt of the invention.

FIG. 4 shows a differential scanning calorimetry (DSC) trace (bottom trace, left side vertical axis) and a thermal gravimetric analysis (TGA) trace (top trace, right side vertical axis) for a crystalline 1-isopropyl-1H-indazole-3-carboxylic acid {(1S,3R,5R)-8-[2-(4-acetylpiperazin-1-yl)ethyl]-8-azabicyclo[3.2.1]oct-3-yl}amide dihydrobromide salt of the invention.

FIG. 5 shows a dynamic moisture sorption (DMS) trace for a crystalline 1-isopropyl-1H-indazole-3-carboxylic acid {(1S,3R,5R)-8-[2-(4-acetylpiperazin-1-yl)ethyl]-8-azabicyclo[3.2.1]oct-3-yl}amide dihydrochloride salt of the invention.

FIG. 6 shows a dynamic moisture sorption (DMS) trace for a crystalline 1-isopropyl-1H-indazole-3-carboxylic acid {(1S,3R,5R)-8-[2-(4-acetylpiperazin-1-yl)ethyl]-8-azabicyclo[3.2.1]oct-3-yl}amide dihydrobromide salt of the invention.

FIG. 7 shows a powder x-ray diffraction (PXRD) pattern of a crystalline hydrate of a 1-isopropyl-1H-indazole-3-carboxylic acid {(1S,3R,5R)-8-[2-(4-acetylpiperazin-1-yl)ethyl]-8-azabicyclo[3.2.1]oct-3-yl}amide dihydrochloride salt of the invention.

FIG. 8 shows a differential scanning calorimetry (DSC) trace (top trace, left side vertical axis) and a thermal gravimetric analysis (TGA) trace (bottom trace, right side vertical axis) for a crystalline hydrate of a 1-isopropyl-1H-indazole-3-carboxylic acid {(1S,3R,5R)-8-[2-(4-acetylpiperazin-1-yl)ethyl]-8-azabicyclo[3.2.1]oct-3-yl}amide dihydrochloride salt of the invention.

### DETAILED DESCRIPTION OF THE INVENTION

The invention provides crystalline halide salts of 1-isopropyl-1H-indazole-3-carboxylic acid {(1S,3R,5R)-8-[2-(4-acetyl-piperazin-1-yl)ethyl]-8-azabicyclo[3.2.1]oct-3-yl}amide and solvates thereof.

Definitions

When describing the compounds, compositions and methods of the invention, the following terms have the following meanings, unless otherwise indicated.

The term “therapeutically effective amount” means an amount sufficient to effect treatment when administered to a patient in need of treatment.

The term “treatment” as used herein means the treatment of a disease, disorder, or medical condition in a patient, such as a mammal (particularly a human) which includes:
(a) preventing the disease, disorder, or medical condition from occurring, i.e., prophylactic treatment of a patient;
(b) ameliorating the disease, disorder, or medical condition, i.e., eliminating or causing regression of the disease, disorder, or medical condition in a patient;
(c) suppressing the disease, disorder, or medical condition, i.e., slowing or arresting the development of the disease, disorder, or medical condition in a patient; or
(d) alleviating the symptoms of the disease, disorder, or medical condition in a patient.

The term “solvate” means a complex or aggregate formed by one or more molecules of a solute, i.e. a compound of the invention or a pharmaceutically-acceptable salt thereof, and one or more molecules of a solvent. Such solvates are typically crystalline solids having a substantially fixed molar ratio of solute and solvent. Representative solvents include by way of example, water, methanol, ethanol, isopropanol, acetic acid, and the like. When the solvent is water, the solvate formed is a hydrate.

The term “crystalline halide salt” as used herein means a crystalline solid that does not include solvent molecules in the crystal lattice, i.e. one that is not a solvate. Solvates, or specifically hydrates, of the invention are identified explicitly.

It must be noted that, as used in the specification and appended claims, the singular forms “a”, “an”, “one”, and “the” may include plural references, unless the content clearly dictates otherwise.

The term “amino-protecting group” means a protecting group suitable for preventing undesired reactions at an amino nitrogen. Representative amino-protecting groups include, but are not limited to, formyl; acyl groups, for example alkanoyl groups, such as acetyl; alkoxycarbonyl groups, such as tert-butoxycarbonyl (Boc); arylmethoxycarbonyl groups, such as benzyloxycarbonyl (Cbz) and 9-fluorenylmethoxycarbonyl (Fmoc); arylmethyl groups, such as benzyl (Bn), trityl (Tr), and 1,1-di-(4′-methoxyphenyl)methyl; silyl groups, such as trimethylsilyl (TMS) and tert-butyldimethylsilyl (TBDMS); and the like.

Active Agent

The active agent in the present salts, i.e. the compound of formula I, is designated 1-isopropyl-1H-indazole-3-carboxylic acid {(1S,3R,5R)-8-[2-(4-acetyl-piperazin-1-yl)ethyl]-8-azabicyclo[3.2.1]oct-3-yl}amide using the commercially-available AutoNom software (MDL Information Systems, GmbH, Frankfurt, Germany). The designation (1S,3R,5R) describes the relative orientation of the bonds associated with the bicyclic ring system. The compound is alternatively denoted as N-[(3-endo)-8-[2-(4-acetyl-piperazin-1-yl)ethyl]-8-azabicyclo[3.2.1]oct-3-yl]-1-(1-methylethyl)-1H-indazole-3-carboxamide.

Halide Salts of the Invention

In one aspect, the invention provides crystalline 1-isopropyl-1H-indazole-3-carboxylic acid {(1S,3R,5R)-8-[2-(4-acetyl-piperazin-1-yl)ethyl]-8-azabicyclo[3.2.1]oct-3-yl}amide dihydrochloride.

A dihydrochloride salt of the invention typically contains between about 1.75 and about 2.2 molar equivalents of hydrochloric acid per molar equivalent of the compound of formula I, including between about 1.9 and about 2.1 molar equivalents of hydrochloric acid per molar equivalent of the compound of formula I.

The molar ratio of a halide acid to the active agent can be readily determined by methods available to those skilled in the art. For example, such molar ratios can be readily determined by elemental analysis. Alternatively, ¹H NMR and ion chromatography methods can be used to determine the molar ratio.

In one aspect, the crystalline dihydrochloride salt of the present invention is characterized by a powder x-ray diffraction (PXRD) pattern having two or more diffraction peaks at 2θ values selected from 4.17±0.20, 8.23±0.20, 12.30±0.20, 12.77±0.20, 13.94±0.20, 16.04±0.20, 17.06±0.20, 18.56±0.20, 19.93±0.20, 20.49±0.20, 21.72±0.20, 22.58±0.20, 26.27±0.20, and 26.59±0.20. In particular, in this aspect, the crystalline form is characterized by a powder x-ray diffraction pattern having two or more diffraction peaks at 2θ values selected from 4.17±0.20, 8.23±0.20, 12.30±0.20, 13.94±0.20, and 18.56±0.20.

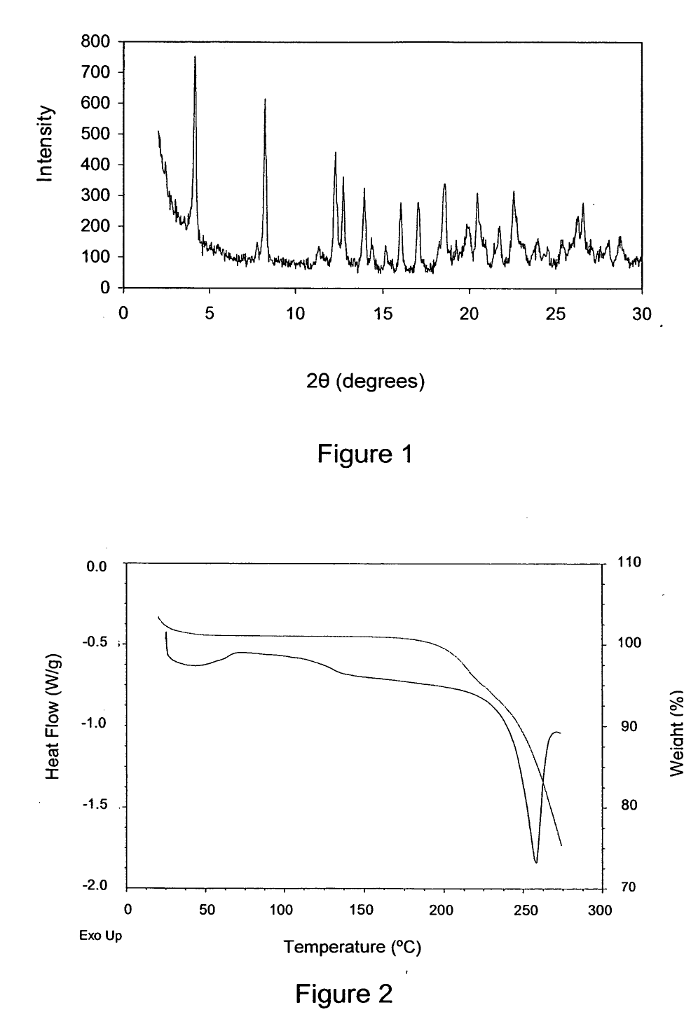

As is well known in the field of powder x-ray diffraction, peak positions of PXRD spectra are relatively less sensitive to experimental details, such as details of sample preparation and instrument geometry, than are the relative peak heights. Thus, in one aspect, a crystalline dihydrochloride salt of the compound of formula I is characterized by a powder x-ray diffraction pattern in which the peak positions are substantially in accordance with those shown in FIG. 1.

The crystalline dihydrochloride salt of the present invention is also characterized high temperature thermal stability as evidenced by its differential scanning calorimetry (DSC) trace which exhibits a peak in endothermic heat flow in the range of about 230° C. to about 275° C., as illustrated in FIG. 2. Furthermore, the thermal gravimetric analysis (TGA) trace shows no significant thermal event below about 180° C.

In yet another aspect a crystalline dihydrochloride salt is characterized by its infrared absorption spectrum which shows significant absorption bands at about 753, 986, 1086, 1197, 1267, 1281, 1437, 1488, 1522, 1638, 1664, and 2416 cm⁻¹.

A crystalline dihydrochloride salt of the compound of formula I has been demonstrated to have a reversible sorption/desorption profile with an acceptable, moderate level of hygroscopicity (i.e., less than about 1% weight gain in the humidity range of 40% relative humidity to 60% relative humidity).

Additionally, the crystalline dihydrochloride salt of the compound of formula I has been found to be stable upon exposure to elevated humidity for an extended period. For example, after storage for six months at 25° C. and 60% relative humidity, analysis by HPLC showed no chemical degradation and less than a 2% gain in moisture content.

In another aspect, the invention provide crystalline 1-isopropyl-1H-indazole-3-carboxylic acid {(1S,3R,5R)-8-[2-(4-acetyl-piperazin-1-yl)ethyl]-8-azabicyclo[3.2.1]oct-3-yl}amide dihydrobromide.

A dihydrobromide salt of the invention typically contains between about 1.75 and about 2.2 molar equivalents of hydrobromic acid per molar equivalent of the compound of formula I.

In one aspect, a crystalline dihydrobromide salt of the present invention is characterized by a powder x-ray diffraction (PXRD) pattern having two or more diffraction peaks at 2θ values selected from 4.41±0.20, 7.63±0.20, 8.75±0.20, 13.11±0.20, 14.52±0.20, 15.28±0.20, 21.89±0.20, 22.75±0.20, 23.35±0.20, 25.40±0.20, 26.34±0.20, 28.53±0.20, and 28.86±0.20. In particular, in this aspect, the crystalline form is characterized by a powder x-ray diffraction pattern having two or more diffraction peaks at 2θ values selected from 4.41±0.20, 8.75±0.20, 13.11±0.20, and 21.89±0.20.

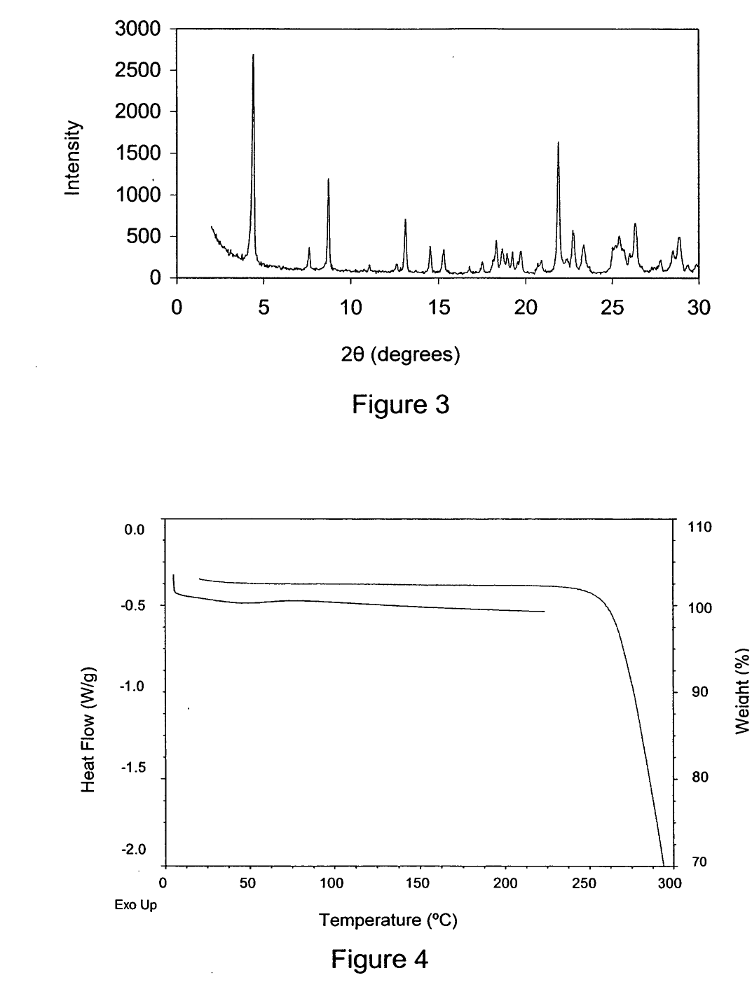

In another aspect, a crystalline dihydrobromide salt of the compound of formula I is characterized by a powder x-ray diffraction pattern in which the peak positions are substantially in accordance with those shown in FIG. 3.

In yet another aspect, a crystalline dihydrobromide salt of the present invention is characterized by exceptional thermal stability as evidenced by its DSC and TGA traces which exhibit no thermal events at temperatures up to about 230° C. and about 250° C., respectively, as illustrated in FIG. 4.

A crystalline dihydrobromide salt of the compound of formula I has been demonstrated to have a reversible sorption/desorption profile with a low level of hygroscopicity (i.e., less than about 1% weight gain in the humidity range of 40% relative humidity to 60% relative humidity).

In yet another aspect, the invention provides a crystalline hydrate of a dihydrochloride salt of the compound of formula I.

In one aspect, a crystalline hydrate of a dihydrochloride salt of the present invention is characterized by a powder x-ray diffraction (PXRD) pattern having two or more diffraction peaks at 2θ values selected from 4.85±0.20, 8.14±0.20, 9.02±0.20, 12.58±0.20, 14.20±0.20, 16.85±0.20, 17.31±0.20, 17.66±0.20, 19.28±0.20, 19.92±0.20, 21.36±0.20, 21.72±0.20, 22.38±0.20, 22.75±0.20, 26.34±0.20, 28.85±0.20, and 29.47±0.20. In particular, in this aspect, the crystalline form is characterized by a powder x-ray diffraction pattern having two or more diffraction peaks at 2θ values selected from 12.58±0.20, 14.20±0.20, 16.85±0.20, 17.31±0.20, and 17.66±0.20.

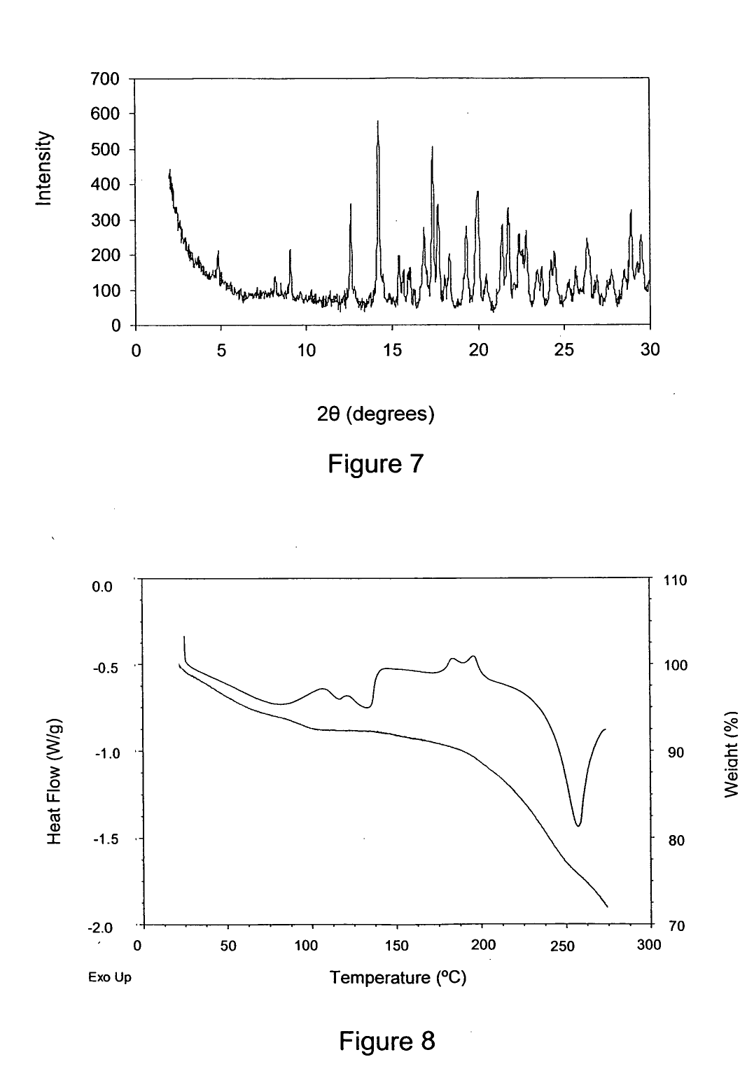

In another aspect, a crystalline hydrate of a dihydrochloride salt of the compound of formula I is characterized by a powder x-ray diffraction pattern in which the peak positions are substantially in accordance with those shown in FIG. 7.

These properties of the salts of this invention are further illustrated in the Examples below.

Synthetic Procedures

To prepare a crystalline salt of the invention, the 1-isopropyl-1H-indazole-3-carboxylic acid {(1S,3R,5R)-8-[2-(4-acetyl-piperazin-1-yl)ethyl]-8-azabicyclo[3.2.1]oct-3-yl}amide is typically contacted with about 1.8 to about 2.3 molar equivalents of a halide acid, such as hydrochloric acid or hydrobromic acid. Generally, this reaction in conducted in an inert diluent at a temperature ranging from about 20° C. to about 70° C., including about 35° C. to about 60° C. Suitable inert diluents for this reaction include, but are not limited to, ethanol, methanol, isopropanol, ethyl acetate, acetonitrile, toluene, dichloroethane, tetrahydrofuran, and combinations thereof, and optionally containing water. In particular aspects, aqueous HCl or aqueous HBr is added to a solution of the active agent in ethanol.

Upon completion of the reaction, a crystalline salt of the invention is isolated from the reaction mixture by any conventional means, such as precipitation, concentration, centrifugation, and the like.

The active agent, 1-isopropyl-1H-indazole-3-carboxylic acid {(1S,3R,5R)-8-[2-(4-acetyl-piperazin-1-yl)ethyl]-8-azabicyclo[3.2.1]oct-3-yl}amide, can be prepared from readily available starting materials using the procedures described in the Examples below, or using the procedures described in the commonly-assigned U.S. applications listed in the Background section of this application.

For example, as described in Example 1B, the active agent can be prepared as illustrated in Scheme A.

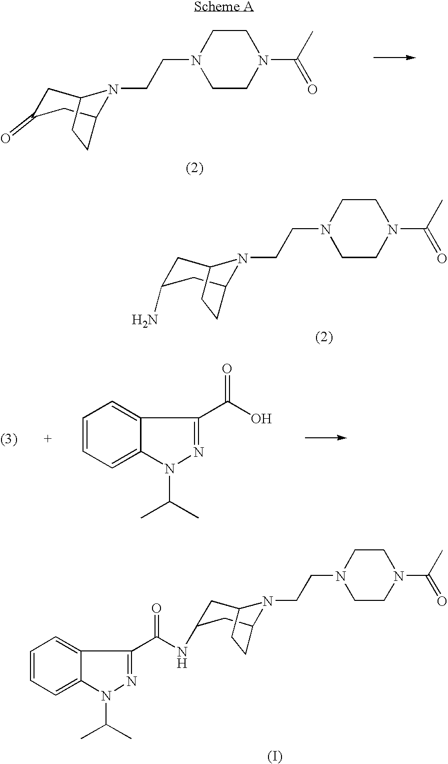

In Scheme A, first (1R,5S)-8-[2-(4-acetylpiperazin 1-yl)ethyl]-8-azabicyclo[3.2.1]-octan-3-one (2) is reacted with ammonium formate in the presence of a catalyst to provide 1-{4-[2-(3-endo-amino-8-azabicyclo[3.2.1]oct-8-yl)ethyl]piperazin-1-yl}ethanone (3). Typically intermediate (2) is reacted with a large excess, for example, at least about 15 equivalents, of ammonium formate in an inert diluent, such as methanol, in the presence of a transition metal catalyst, for example, palladium, to provide intermediate (3) in the endo configuration with high stereospecificity. The reaction is typically conducted at ambient temperature for about 12 to about 72 hours or until the reaction is substantially complete. The product can be purified by conventional procedures, such as by extraction.

In the second step, aminotropane (3) is reacted with 1H-indazole carboxylic acid (4) in the presence of a coupling agent and a base to provide the compound of formula (I). Typically, the aminotropane (3), diluted in an inert diluent, such as acetonitrile, is combined with the carboxylic acid (4) diluted in an inert diluent, such as acetonitrile, in the presence of a base, such as triethylamine, diisopropylethylamine, and the like, and a coupling agent, such as benzotriazol-1-yloxytripyrrolidino-phosphonium hexafluoro-phosphate (PyBop), or 1,3-dicyclohexyl-carbodiimide (DCC), 1-(3-dimethylamino-propyl)-3-ethylcarbodiimide (EDC), or the like. The reaction is typically initially conducted at a temperature of from about 0° C. to about 10° C. for about 30 min to about 1 h, then at room temperature for about 1 to 3 h or until the reaction is substantially complete. The product can be isolated by conventional procedures.

As described in Example 1B herein, intermediate (2) can be prepared from commonly available starting materials.

Further details regarding specific reaction conditions and other procedures for preparing a compound of formula (I) or intermediates thereto are described in the examples below.

Accordingly, in another aspect, the invention provides a process for preparing a compound of formula (I), the process comprising:
(a) reacting intermediate (2) with an excess of ammonium formate in the presence of a transition metal catalyst, to provide intermediate (3); and
(b) reacting intermediate (3) with intermediate (4) in the presence of a coupling agent and a base, to provide a compound of formula (1), or a pharmaceutically-acceptable salt or solvate or stereoisomer thereof.

The invention further provides novel intermediates (2) and (3).

Pharmaceutical Compositions

The crystalline halide salts of the compound of formula I are typically administered to a patient in the form of a pharmaceutical composition. Such pharmaceutical compositions may be administered to the patient by any acceptable route of administration including, but not limited to, oral, rectal, vaginal, nasal, inhaled, topical (including transdermal) and parenteral modes of administration.

Accordingly, in one of its compositions aspects, the invention is directed to a pharmaceutical composition comprising a pharmaceutically-acceptable carrier or excipient and a therapeutically effective amount of a halide salt of a compound of formula I. Optionally, such pharmaceutical compositions may contain other therapeutic and/or formulating agents if desired.

The pharmaceutical compositions of the invention typically contain a therapeutically effective amount of a crystalline salt of the present invention. Typically, such pharmaceutical compositions will contain from about 0.1 to about 95% by weight of the active agent; including from about 1 to about 70% by weight, such as from about 5 to about 60% by weight of the active agent.

Any conventional carrier or excipient may be used in the pharmaceutical compositions of the invention. The choice of a particular carrier or excipient, or combinations of carriers or excipients, will depend on the mode of administration being used to treat a particular patient or type of medical condition or disease state. In this regard, the preparation of a suitable pharmaceutical composition for a particular mode of administration is well within the scope of those skilled in the pharmaceutical arts. Additionally, the ingredients for such compositions are commercially-available from, for example, Sigma, P.O. Box 14508, St. Louis, Mo. 63178. By way of further illustration, conventional formulation techniques are described in Remington: The Science and Practice of Pharmacy, 20^{th} Edition, Lippincott Williams & White, Baltimore, Md. (2000); and H. C. Ansel et al., Pharmaceutical Dosage Forms and Drug Delivery Systems, 7^{th} Edition, Lippincott Williams & White, Baltimore, Md. (1999).

Representative examples of materials which can serve as pharmaceutically acceptable carriers include, but are not limited to, the following: (1) sugars, such as lactose, glucose and sucrose; (2) starches, such as corn starch and potato starch; (3) cellulose, such as microcrystalline cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical compositions.

The pharmaceutical compositions of the invention are typically prepared by thoroughly and intimately mixing or blending a compound of the invention with a pharmaceutically-acceptable carrier and one or more optional ingredients. If necessary or desired, the resulting uniformly blended mixture can then be shaped or loaded into tablets, capsules, pills and the like using conventional procedures and equipment.

The pharmaceutical compositions of the invention are preferably packaged in a unit dosage form. The term “unit dosage form” refers to a physically discrete unit suitable for dosing a patient, i.e., each unit containing a predetermined quantity of active agent calculated to produce the desired therapeutic effect either alone or in combination with one or more additional units. For example, such unit dosage forms may be capsules, tablets, pills, and the like.

In a preferred embodiment, the pharmaceutical compositions of the invention are suitable for oral administration. Suitable pharmaceutical compositions for oral administration may be in the form of capsules, tablets, pills, lozenges, cachets, dragees, powders, granules; or as a solution or a suspension in an aqueous or non-aqueous liquid; or as an oil-in-water or water-in-oil liquid emulsion; or as an elixir or syrup; and the like; each containing a predetermined amount of a compound of the present invention as an active ingredient.

When intended for oral administration in a solid dosage form (i.e., as capsules, tablets, pills and the like), the pharmaceutical compositions of the invention will typically comprise a compound of the present invention as the active ingredient and one or more pharmaceutically-acceptable carriers, such as sodium citrate or dicalcium phosphate. Optionally or alternatively, such solid dosage forms may also comprise: (1) fillers or extenders, such as starches, microcrystalline cellulose, lactose, sucrose, glucose, mannitol, and/or silicic acid; (2) binders, such as carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and/or sodium carbonate; (5) solution retarding agents, such as paraffin; (6) absorption accelerators, such as quaternary ammonium compounds; (7) wetting agents, such as cetyl alcohol and/or glycerol monostearate; (8) absorbents, such as kaolin and/or bentonite clay; (9) lubricants, such as talc, calcium stearate, magnesium stearate, solid polyethylene glycols, sodium lauryl sulfate, and/or mixtures thereof; (10) coloring agents; and (11) buffering agents.

Release agents, wetting agents, coating agents, sweetening, flavoring and perfuming agents, preservatives and antioxidants can also be present in the pharmaceutical compositions of the invention. Examples of pharmaceutically-acceptable antioxidants include: (1) water-soluble antioxidants, such as ascorbic acid, cysteine hydrochloride, sodium bisulfate, sodium metabisulfate sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like. Coating agents for tablets, capsules, pills and like, include those used for enteric coatings, such as cellulose acetate phthalate (CAP), polyvinyl acetate phthalate (PVAP), hydroxypropyl methylcellulose phthalate, methacrylic acid-methacrylic acid ester copolymers, cellulose acetate trimellitate (CAT), carboxymethyl ethyl cellulose (CMEC), hydroxypropyl methyl cellulose acetate succinate (HPMCAS), and the like.

If desired, the pharmaceutical compositions of the present invention may also be formulated to provide slow or controlled release of the active ingredient using, by way of example, hydroxypropyl methyl cellulose in varying proportions; or other polymer matrices, liposomes and/or microspheres.

In addition, the pharmaceutical compositions of the present invention may optionally contain opacifying agents and may be formulated so that they release the active ingredient only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Suitable liquid dosage forms for oral administration include, by way of illustration, pharmaceutically-acceptable emulsions, microemulsions, solutions, suspensions, syrups and elixirs. Such liquid dosage forms typically comprise the active ingredient and an inert diluent, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (esp., cottonseed, groundnut, corn, germ, olive, castor and sesame oils), glycerol, tetrahydrofuryl alcohol, polyethylene glycols and fatty acid esters of sorbitan, and mixtures thereof. Suspensions, in addition to the active ingredient, may contain suspending agents such as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Alternatively, the pharmaceutical compositions of the invention are formulated for administration by inhalation. Suitable pharmaceutical compositions for administration by inhalation will typically be in the form of an aerosol or a powder. Such compositions are generally administered using well-known delivery devices, such as a metered-dose inhaler, a dry powder inhaler, a nebulizer or a similar delivery device.

When administered by inhalation using a pressurized container, the pharmaceutical compositions of the invention will typically comprise the active ingredient and a suitable propellant, such as dichlorodifluoromethane, trichlorofluoromethane, dichlorotetrafluoroethane, carbon dioxide or other suitable gas.

Additionally, the pharmaceutical composition may be in the form of a capsule or cartridge (made, for example, from gelatin) comprising a compound of the invention and a powder suitable for use in a powder inhaler. Suitable powder bases include, by way of example, lactose or starch.

The compounds of the invention can also be administered transdermally using known transdermal delivery systems and excipients. For example, a compound of the invention can be admixed with permeation enhancers, such as propylene glycol, polyethylene glycol monolaurate, azacycloalkan-2-ones and the like, and incorporated into a patch or similar delivery system. Additional excipients including gelling agents, emulsifiers and buffers, may be used in such transdermal compositions if desired.

The following formulations illustrate representative pharmaceutical compositions of the present invention:

### FORMULATION EXAMPLE A

Hard gelatin capsules for oral administration are prepared as follows:

| Ingredients | Amount | |
|---|---|---|
| Salt of the invention | 50 | mg |
| Lactose (spray-dried) | 200 | mg |
| Magnesium stearate | 10 | mg |

Representative Procedure: The ingredients are thoroughly blended and then loaded into a hard gelatin capsule (260 mg of composition per capsule).

### FORMULATION EXAMPLE B

Hard gelatin capsules for oral administration are prepared as follows:

| Ingredients | Amount | |
|---|---|---|
| Salt of the invention | 20 | mg |
| Starch | 89 | mg |
| Microcrystalline cellulose | 89 | mg |
| Magnesium stearate | 2 | mg |

Representative Procedure: The ingredients are thoroughly blended and then passed through a No. 45 mesh U.S. sieve and loaded into a hard gelatin capsule (200 mg of composition per capsule).

### FORMULATION EXAMPLE C

Capsules for oral administration are prepared as follows:

| Ingredients | Amount | |
|---|---|---|
| Salt of the invention | 10 | mg |
| Polyoxyethylene sorbitan monooleate | 50 | mg |
| Starch powder | 250 | mg |

Representative Procedure: The ingredients are thoroughly blended and then loaded into a gelatin capsule (310 mg of composition per capsule).

### FORMULATION EXAMPLE D

Tablets for oral administration are prepared as follows:

| Ingredients | Amount | |
|---|---|---|
| Salt of the invention | 5 | mg |
| Starch | 50 | mg |
| Microcrystalline cellulose | 35 | mg |
| Polyvinylpyrrolidone | 4 | mg |
| (10 wt. % in water) | | |
| Sodium carboxymethyl starch | 4.5 | mg |
| Magnesium stearate | 0.5 | mg |
| Talc | 1 | mg |

Representative Procedure: The active ingredient, starch and cellulose are passed through a No. 45 mesh U.S. sieve and mixed thoroughly. The solution of polyvinylpyrrolidone is mixed with the resulting powders, and this mixture is then passed through a No. 14 mesh U.S. sieve. The granules so produced are dried at 50-60° C. and passed through a No. 18 mesh U.S. sieve. The sodium carboxymethyl starch, magnesium stearate and talc (previously passed through a No. 60 mesh U.S. sieve) are then added to the granules. After mixing, the mixture is compressed on a tablet machine to afford a tablet weighing 100 mg.

### FORMULATION EXAMPLE E

Tablets for oral administration are prepared as follows:

| Ingredients | Amount | |
|---|---|---|
| Salt of the invention | 25 | mg |
| Microcrystalline cellulose | 400 | mg |
| Silicon dioxide fumed | 10 | mg |
| Stearic acid | 5 | mg |

Representative Procedure: The ingredients are thoroughly blended and then compressed to form tablets (440 mg of composition per tablet).

### FORMULATION EXAMPLE F

Single-scored tablets for oral administration are prepared as follows:

| Ingredients | Amount | |
|---|---|---|
| Salt of the invention | 15 | mg |
| Cornstarch | 50 | mg |
| Croscarmellose sodium | 25 | mg |
| Lactose | 120 | mg |
| Magnesium stearate | 5 | mg |

Representative Procedure: The ingredients are thoroughly blended and compressed to form a single-scored tablet (215 mg of compositions per tablet).

### FORMULATION EXAMPLE G

A suspension for oral administration is prepared as follows:

| Ingredients | Amount | |
|---|---|---|
| Salt of the invention | 0.1 | g |
| Fumaric acid | 0.5 | g |
| Sodium chloride | 2.0 | g |
| Methyl paraben | 0.15 | g |
| Propyl paraben | 0.05 | g |
| Granulated sugar | 25.5 | g |
| Sorbitol (70% solution) | 12.85 | g |
| Veegum k (Vanderbilt Co.) | 1.0 | g |
| Flavoring | 0.035 | mL |
| Colorings | 0.5 | mg |
| Distilled water | q.s. to 100 | mL |

Representative Procedure: The ingredients are mixed to form a suspension containing 10 mg of active ingredient per 10 mL of suspension.

### FORMULATION EXAMPLE H

A dry powder for administration by inhalation is prepared as follows:

| Ingredients | Amount | |
|---|---|---|
| Salt of the invention | 1.0 | mg |
| Lactose | 25 | mg |

Representative Procedure: The active ingredient is micronized and then blended with lactose. This blended mixture is then loaded into a gelatin inhalation cartridge. The contents of the cartridge are administered using a powder inhaler.

### FORMULATION EXAMPLE I

A dry powder for administration by inhalation in a metered dose inhaler is prepared as follows:

Representative Procedure: A suspension containing 5 wt. % of a salt of the invention and 0.1 wt. % lecithin is prepared by dispersing 10 g of active compound as micronized particles with mean size less than 10 g/m in a solution formed from 0.2 g of lecithin dissolved in 200 mL of demineralized water. The suspension is spray dried and the resulting material is micronized to particles having a mean diameter less than 1.5 μm. The particles are loaded into cartridges with pressurized 1,1,1,2-tetrafluoroethane.

### FORMULATION EXAMPLE J

An injectable formulation is prepared as follows:

| Ingredients | Amount | |
|---|---|---|
| Salt of the invention | 0.2 | g |
| Sodium acetate buffer | 40 | mL |
| solution (0.4 M) | | |
| HCl (0.5 N) or NaOH (0.5 N) | q.s. to pH | 4 |
| Water (distilled, sterile) | q.s. to 20 | mL |

Representative Procedure: The above ingredients are blended and the pH is adjusted to 4±0.5 using 0.5 N HCl or 0.5 N NaOH.

### FORMULATION EXAMPLE K

Capsules for oral administration are prepared as follows:

| Ingredients | Amount | |
|---|---|---|
| Salt of the Invention | 4.05 | mg |
| Microcrystalline cellulose | 259.2 | mg |
| (Avicel PH 103) | | |
| Magnesium stearate | 0.75 | mg |

Representative Procedure: The ingredients are thoroughly blended and then loaded into a gelatin capsule (Size #1, White, Opaque) (264 mg of composition per capsule).

### FORMULATION EXAMPLE L

Capsules for oral administration are prepared as follows:

| Ingredients | Amount | |
|---|---|---|
| Salt of the Invention | 8.2 | mg |
| Microcrystalline cellulose | 139.05 | mg |
| (Avicel PH 103) | | |
| Magnesium stearate | 0.75 | mg |

Representative Procedure: The ingredients are thoroughly blended and then loaded into a gelatin capsule (Size #1, White, Opaque) (148 mg of composition per capsule).

Utility

The compound of formula I, 1-isopropyl-1H-indazole-3-carboxylic acid {(1S,3R,5R)-8-[2-(4-acetyl-piperazin-1-yl)ethyl]-8-azabicyclo[3.2.1]oct-3-yl}amide, is a 5-HT₄ receptor agonist and therefore the present crystalline halide salt forms of the compound of formula I are expected to be useful for treating medical conditions mediated by 5-HT₄ receptors or associated with 5-HT₄ receptor activity, i.e. medical conditions which are ameliorated by treatment with a 5-HT₄ receptor agonist. Such medical conditions include, but are not limited to, irritable bowel syndrome (IBS), chronic constipation, functional dyspepsia, delayed gastric emptying, gastroesophageal reflux disease (GERD), gastroparesis, diabetic and idiopathic gastropathy, post-operative ileus, intestinal pseudo-obstruction, and drug-induced delayed transit. In addition, it has been suggested that some 5-HT₄ receptor agonist compounds may be used in the treatment of central nervous system disorders including cognitive disorders, behavioral disorders, mood disorders, and disorders of control of autonomic function.

In particular, the salts of the invention increase motility of the gastrointestinal (GI) tract and thus are expected to be useful for treating disorders of the GI tract caused by reduced motility in mammals, including humans. Such GI motility disorders include, by way of illustration, chronic constipation, constipation-predominant irritable bowel syndrome (C-IBS), diabetic and idiopathic gastroparesis, and functional dyspepsia.

In one aspect, therefore, the invention provides a method of increasing motility of the gastrointestinal tract in a mammal, the method comprising administering to the mammal a therapeutically effective amount of a pharmaceutical composition comprising a pharmaceutically-acceptable carrier and a crystalline salt of the invention.

When used to treat disorders of reduced motility of the GI tract or other conditions mediated by 5-HT₄ receptors, the salts of the invention will typically be administered orally in a single daily dose or in multiple doses per day, although other forms of administration may be used. The amount of active agent administered per dose or the total amount administered per day will typically be determined by a physician, in the light of the relevant circumstances, including the condition to be treated, the chosen route of administration, the actual compound administered and its relative activity, the age, weight, and response of the individual patient, the severity of the patient's symptoms, and the like.

Suitable doses for treating disorders of reduced motility of the GI tract or other disorders mediated by 5-HT₄ receptors are expected to range from about 0.0007 to about 20 mg/kg/day of active agent, including from about 0.0007 to about 1 mg/kg/day. For an average 70 kg human, this would amount to from about 0.05 to about 70 mg per day of active agent.

In one aspect of the invention, the salts of the invention are used to treat chronic constipation. When used to treat chronic constipation, the salts of the invention will typically be administered orally in a single daily dose or in multiple doses per day. The dose for treating chronic constipation is expected to range from about 0.05 to about 70 mg per day.

In another aspect of the invention, the salts of the invention are used to treat irritable bowel syndrome. When used to treat constipation-predominant irritable bowel syndrome, the salts of the invention will typically be administered orally in a single daily dose or in multiple doses per day. The dose for treating constipation-predominant irritable bowel syndrome is expected to range from about 0.05 to about 70 mg per day.

In another aspect of the invention, the salts of the invention are used to treat diabetic and idiopathic gastroparesis. When used to treat diabetic and idiopathic gastroparesis, the salts of the invention will typically be administered orally in a single daily dose or in multiple doses per day. The dose for treating diabetic gastroparesis is expected to range from about 0.05 to about 70 mg per day.

In yet another aspect of the invention, the salts of the invention are used to treat functional dyspepsia. When used to treat functional dyspepsia, the compounds of the invention will typically be administered orally in a single daily dose or in multiple doses per day. The dose for treating functional dyspepsia is expected to range from about 0.05 to about 70 mg per day.

The invention also provides a method of treating a mammal having a disease or condition associated with 5-HT₄ receptor activity, the method comprising administering to the mammal a therapeutically effective amount of a salt of the invention or of a pharmaceutical composition comprising a salt of the invention.

As described above, salts of the invention are 5-HT₄ receptor agonists. The invention further provides, therefore, a method of agonizing a 5-HT₄ receptor in a mammal, the method comprising administering a salt of the invention to the mammal.

There properties, as well as the utility of the halide salts of the invention, can be demonstrated using various in vitro and in vivo assays well-known to those skilled in the art. Representative assays are described in further detail in the following examples.

### EXAMPLES

The following synthetic and biological examples are offered to illustrate the invention, and are not to be construed in any way as limiting the scope of the invention. In the examples below, the following abbreviations have the following meanings unless otherwise indicated. Abbreviations not defined below have their generally accepted meanings.
Boc=tert-butoxycarbonyl
(Boc)₂O=di-tert-butyl dicarbonate
DCM=dichloromethane
DMF=N,N-dimethylformamide
DMSO=dimethyl sulfoxide
EtOAc=ethyl acetate
mCPBA=m-chlorobenzoic acid
MeCN=acetonitrile
MTBE=tert-butyl methyl ether
PyBop=benzotriazol-1-yloxytripyrrolidino-phosphonium hexafluorophosphate
R_{f}=retention factor
RT=room temperature
TFA=trifluoroacetic acid
THF=tetrahydrofuran

Reagents (including secondary amines) and solvents were purchased from commercial suppliers (Aldrich, Fluka, Sigma, etc.), and used without further purification. Reactions were run under nitrogen atmosphere, unless noted otherwise. Progress of reaction mixtures was monitored by thin layer chromatography (TLC), analytical high performance liquid chromatography (anal. HPLC), and mass spectrometry, the details of which are given below and separately in specific examples of reactions. Reaction mixtures were worked up as described specifically in each reaction; commonly they were purified by extraction and other purification methods such as temperature-, and solvent-dependent crystallization, and precipitation. In addition, reaction mixtures were routinely purified by preparative HPLC: a general protocol is described below. Characterization of reaction products was routinely carried out by mass and ¹H-NMR spectrometry. For NMR measurement, samples were dissolved in deuterated solvent (CD₃OD, CDCl₃, or DMSO-d₆), and ¹H-NMR spectra were acquired with a Varian Gemini 2000 instrument (300 MHz) under standard observation conditions. Mass spectrometric identification of compounds was performed by an electrospray ionization method (ESMS) with an Applied Biosystems (Foster City, Calif.) model API 150 EX instrument or an Agilent (Palo Alto, Calif.) model 1100 LC/MSD instrument.

General Protocol for Analytical HPLC

Crude compounds were dissolved in 50% MeCN/H₂O (with 0.1% TFA) at 0.5-1.0 mg/mL concentration, and analyzed using the following conditions:

Column: Zorbax Bonus-RP (3.5 μm of particle size, 2.1×50 mm)

Flow rate: 0.5 mL/min

Detector wavelength: 214, 254, and 280 nm.

General Protocol for Preparative HPLC Purification

Crude compounds were dissolved in 50% acetic acid in water at 50-100 mg/mL concentration, filtered, and fractionated using the following procedure:

Column: YMC Pack-Pro C18 (50a×20 mm; ID=5 μm)

Flow rate: 40 mL/min

Mobile Phases: A=90% MeCN/10% H₂O/0.1% TFA
B=98% H₂O/2% MeCN/0.1% TFA

Gradient: 10% A/90% B to 50% A/50% B over 30 min (linear)

Detector wavelength: 214 nm.

### Preparation 1: (1S,3R,5R)-3-amino-8-azabicyclo[3.2.1]octane-8-carboxylic acid tert-butyl ester

### a. Preparation of 8-benzyl-8-azabicyclo[3.2.1]octan-3-one

Concentrated hydrochloric acid (30 mL) was added to a heterogeneous solution of 2,5-dimethoxy tetrahydrofuran (82.2 g, 0.622 mol) in water (170 mL) while stirring. In a separate flask cooled to 0° C. (ice bath), concentrated hydrochloric acid (92 mL) was added slowly to a solution of benzyl amine (100 g, 0.933 mol) in water (350 mL). The 2,5-dimethoxytetrahydrofuran solution was stirred for approximately 20 min, diluted with water (250 mL), and then the benzyl amine solution was added, followed by the addition of a solution of 1,3-acetonedicarboxylic acid (100 g, 0.684 mol) in water (400 mL) and then the addition of sodium hydrogen phosphate (44 g, 0.31 mol) in water (200 mL). The pH was adjusted from pH 1 to pH ˜4.5 using 40% NaOH. The resulting cloudy and pale yellow solution was stirred overnight. The solution was then acidified to pH 3 from pH 7.5 using 50% hydrochloric acid, heated to 85° C. and stirred for 2 hours. The solution was cooled to room temperature, basified to pH 12 using 40% NaOH, and extracted with dichloromethane (3×500 mL). The combined organic layers were washed with brine, dried (MgSO₄), filtered and concentrated under reduced pressure to produce the crude title intermediate as a viscous brown oil.

To a solution of the crude intermediate in methanol (1000 mL) was added di-tert-butyl dicarbonate (74.6 g, 0.342 mol) at 0° C. The solution was allowed to warm to room temperature and stirred overnight. The methanol was removed under reduced pressure and the resulting oil was dissolved in dichloromethane (1000 mL). The intermediate was extracted into 1 M H₃PO₄ (1000 mL) and washed with dichloromethane (3×250 mL) The aqueous layer was basified to pH 12 using aqueous NaOH, and extracted with dichloromethane (3×500 mL). The combined organic layers were dried (MgSO₄), filtered and concentrated under reduced pressure to produce the title intermediate as a viscous, light brown oil. ¹H-NMR (CDCl₃) δ (ppm) 7.5-7.2 (m, 5H, C₆H₅), 3.7 (s, 2H, CH₂Ph), 3.45 (broad s, 2H, CH-NBn), 2.7-2.6 (dd, 2H, CH₂CO), 2.2-2.1 (dd, 2H, CH₂CO), 2.1-2.0 (m, 2H, CH₂CH₂), 1.6 (m, 2H, CH₂CH₂). (m/z): [M+H]⁺ calcd for C₁₄H₁₇NO 216.14; found, 216.0.

### b. Preparation of 3-oxo-8-azabicyclo[3.2.1]octane-8-carboxylic Acid tert-butyl ester

To a solution of 8-benzyl-8-azabicyclo[3.2.1]octan-3-one (75 g, 0.348 mol) in EtOAc (300 mL) was added a solution of di-tert-butyl dicarbonate (83.6 g, 0.383 mol, 1.1 eq) in EtOAc (300 mL). The resulting solution and rinse (100 mL EtOAc) was added to a 1 L Parr hydrogenation vessel containing 23 g of palladium hydroxide (20 wt. % Pd, dry basis, on carbon, ˜50% wet with water; e.g. Pearlman's catalyst) under a stream of nitrogen. The reaction vessel was degassed (alternating vacuum and N₂ five times) and pressurized to 60 psi of H₂ gas. The reaction solution was agitated for two days and recharged with H₂ as needed to keep the H₂ pressure at 60 psi until the reaction was complete as monitored by silica thin layer chromatography. The black solution was then filtered through a pad of Celite® and concentrated under reduced pressure to yield the title intermediate quantitatively as a viscous, yellow to orange oil. It was used in the next step without further treatment. ¹H NMR (CDCl₃) δ (ppm) 4.5 (broad, 2H, CH-NBoc), 2.7 (broad, 2H, CH₂CO), 2.4-2.3 (dd, 2H, CH₂CH₂), 2.1 (broad m, 2H, CH₂CO), 1.7-1.6 (dd, 2H, CH₂CH₂), 1.5 (s, 9H, (CH₃)₃COCON)).

### c. Preparation of (1S,3R,5R)-3-amino-8-azabicyclo[3.2.1]octane-8-carboxylic Acid tert-butyl ester

To a solution of the product of the previous step (75.4 g, 0.335 mol) in methanol (1 L) was added ammonium formate (422.5 g, 6.7 mol), water (115 mL) and 65 g of palladium on activated carbon (10% on dry basis, ˜50% wet with water; Degussa type E101NE/W) under a stream of N₂ while stirring via mechanical stirrer. After 24 and 48 hours, additional portions of ammonium formate (132 g, 2.1 mol) were added each time. Once reaction progression ceased, as monitored by anal. HPLC, Celite® (>500 g) was added and the resulting thick suspension was filtered and then the collected solid was rinsed with methanol (˜500 mL). The filtrates were combined and concentrated under reduced pressure until all methanol had been removed. The resulting cloudy, biphasic solution was then diluted with 1M phosphoric acid to a final volume of ˜1.5 to 2.0 L at pH 2 and washed with dichloromethane (3×700 mL). The aqueous layer was basified to 5 pH 12 using 40% aq. NaOH, and extracted with dichloromethane (3×700 mL). The combined organic layers were dried over MgSO₄, filtered, and concentrated by rotary evaporation, then high-vacuum leaving 52 g (70%) of the title intermediate, commonly N-Boc-endo-3-aminotropane, as a white to pale yellow solid. The isomer ratio of endo to exo amine of the product was >99 based on ¹H-NMR analysis (>96% purity by analytical HPLC). ¹H NMR (CDCl₃) δ (ppm) 4.2-4.0 (broad d, 2H, CHNBoc), 3.25 (t, 1H, CHNH₂), 2.1-2.05 (m, 4H), 1.9 (m, 2H), 1.4 (s, 9H, (CH₃)₃OCON), 1.2-1.1 (broad, 2H). (m/z): [M+H]⁺ calcd for C₁₂H₂₂N₂O₂ 227.18; found, 227.2. Analytical HPLC (isocratic method; 2:98 (A:B) to 90:10 (A:B) over 5 min): retention time=2.14 min.

### Preparation 2: 1-isopropyl-1H-indazole-3-carboxylic Acid

To indazole-3-carboxylic acid (40 g, 247 mmol) suspended in methanol (700 mL) was added concentrated H₂SO₄ (10 mL) slowly while stirring the mixture. The mixture was stirred and refluxed at 80° C. for 24 h. The mixture was cooled, filtered, and concentrated under reduced pressure to afford a pale yellow solid. The solid was suspended in water (700 mL), crushed to fine powder, collected by filtration, and rinsed with water (˜400 mL). The product was suspended in toluene, and evaporated to dryness under reduced pressure, affording indazole-3-carboxylic acid methyl ester as a pale yellow solid (45 g, >95% pure).

(m/z): [M+H]⁺ calcd for C₉H₈N₂O₂ 177.07; found, 177.0. ¹H-NMR (CD₃OD, 300 MHz): δ (ppm) 8.0 (1H, d), 7.5 (1H, d), 7.4 (1H, t), 7.2 (1H, t), 3.9 (3H, s).

To a solution of indazole-3-carboxylic acid methyl ester (40.7 g, 231 mmol) in anhydrous tetrahydrofuran (700 mL) cooled in an ice bath was added slowly solid potassium tert-butoxide (28.3 g, 252 mmol). The mixture was stirred at the same temperature for 1 hr prior to the addition of 2-iododopropane (34.4 mL, 367 mmol). The final mixture was stirred for 12 h at ambient temperature, and refluxed for 12 h. After cooling to room temperature, the mixture was filtered, and the collected solid was rinsed with tetrahydrofuran (100 mL). The filtrates were combined, and concentrated to dryness under reduced pressure, affording crude 1-isopropyl-1H-indazole-3-carboxylic acid methyl ester (49.7 g) as a pale yellow oil. The crude material was purified by flash silica gel chromatography eluting with hexane/ethylacetate (9/1 to 3/1) to yield 1-isopropyl-1H-indazole-3-carboxylic acid methyl ester (43 g, 197 mmol, >99% pure). ¹H-NMR (CD₃OD, 300 MHz): δ (ppm) 8.1-8.0 (1H, d), 7.6 (1H, d), 7.4 (1H, t), 7.2 (1H, t), 5.0 (1H, quin), 3.9 (s, 3H), 1.5 (6H, d).

To a solution of the methyl ester dissolved in tetrahydrofuran (400 mL) was added 1M NaOH (400 mL). The mixture was stirred for 24 h at ambient temperature. The reaction was terminated by washing with ethylacetate (2×400 mL), saving the aqueous layer. The aqueous layer was acidified slowly by adding conc. HCl (˜40 mL) in an ice bath, which led to separation of a pale yellow oily product. The product was extracted with ethylacetate (1000 mL), and the organic layer was dried over MgSO₄ and evaporated under reduced pressure to yield the title intermediate as a pale yellow to white solid (34 g, >98% pure), which was further purified by crystallization from ethylacetate to provide the title intermediate as colorless needles. (m/z): [M+Na]⁺ calcd for C₁₁H₁₂N₂O₂ 226.07; found, 226.6. ¹H-NMR (CD₃OD, 300 MHz):): δ (ppm) 8.1-8.0 (1H, d), 7.6 (1H, d), 7.4 (1H, t), 7.2 (1H, t), 5.0 (1H, quin), 1.5 (6H, d).

### Example 1A

### Synthesis of 1-isopropyl-1H-indazole-3-carboxylic Acid {(1S,3R,5R)-8-[2-(4-acetylpiperazin-1-yl)ethyl]-8-azabicyclo[3.2.1]oct-3-yl}amide dihydrochloride

### a. Preparation of (1S,3R,5R)-3-[1-isopropyl-1H-indazole-3-carbonyl)amino]-8-azabicyclo[3.2.1]octane-8-carboxylic Acid tert-butyl ester

A 5 L three-necked round bottom flask equipped with a magnetic stir bar, a reflux condenser, an addition funnel, a nitrogen inlet and a thermometer was charged with 1-isopropyl-1H-indazole-3-carboxylic acid (250 g, 1.224 mol, 1.1 eq) and 2.5 L of toluene. The resulting suspension was stirred and heated at 70-80° C. To this suspension was added thionyl chloride (218.4 g, 1.836 mol, 1.65 eq) over a period of 40 min. The mixture was heated at 90-100° C. for 1 h and was cooled to 25° C.

A separate 12 L three-necked round bottom flask equipped with a mechanical stirrer, an addition funnel, a nitrogen inlet and a thermometer was charged with 2.5 L of toluene and 3 N NaOH (prepared from diluting 356 g of 50% NaOH with water to 1.48 L), and (1S,3R,5R)-3-amino-8-azabicyclo[3.2.1]octane-8-carboxylic acid tert-butyl ester (251.9 g, 1.113 mol, 1 eq). The resulting suspension was stirred at 23° C. for 10 min and was cooled to 5° C. To this suspension was added the acid chloride solution in toluene over a period of 90 min keeping the internal temperature at ˜5° C. throughout the addition period. The mixture was stirred for 30 min. The reaction was warmed to 25° C.; the aqueous layer was discarded (1.58 L, pH>13). The organic layer was washed with 1 L of 20 wt % brine; and the aqueous layer was discarded (1.005 L, ˜pH 8). The organic layer was collected (5.3 L) and was concentrated to half of the volume (˜2.6 L), and was used in the following step without purification

### b. Preparation of 1-isopropyl-1H-indazole-3-carboxylic Acid {(1S,3R,5R)-8-aza-bicyclo[3.2.1]oct-3-yl}amide

A 12 L three-necked round bottom flask equipped with a mechanical stirrer, an addition funnel, a nitrogen inlet and a thermometer was charged with the product of the previous step. To this solution was added trifluoroacetic acid (0.65 L) over a period of 10 min. The resulting mixture was stirred for 1 h at ambient temperature.

Water (3.3 L) was added to the reaction mixture. The resulting suspension was stirred at 23° C. for 10 min and was allowed to settle to give a three-layer mixture. The top two layers were discarded and the bottom layer (820 mL) was collected and added to MTBE (6560 mL) over a period of 90 min. The resulting suspension was cooled to 5° C. and was agitated for 1 h. The suspension was filtered; the wet cake was washed with MTBE (500 mL), and dried under reduced pressure (80 mm Hg) for 60 h to give the title intermediate (386 g, 81% yield, 99.2% purity by HPLC) as an off-white sandy solid.

### c. Preparation of 1-isopropyl-1H-indazole-3-carboxylic Acid {(1S,3R,5R)-8-(2,2-dimethoxyethyl)-8-aza-bicyclo[3.2.1]oct-3-yl}amide

A 3 L three-necked round bottom flask equipped with a magnetic stirrer, a nitrogen inlet and a thermometer was charged with the intermediate of the previous step (84 g, 0.197 mol), dichloromethane (840 mL), and sodium triacetoxyborohydride (62.6 g, 0.295 mol). The resulting suspension was stirred for 10 min, cooled to 10° C. and 60 wt % aqueous dimethoxyacetaldehyde (51.3 g, 0.295 mol) was added. This solution was stirred for 30 min, warmed to 25° C., and stirred for 1 h. The mixture was filtered through Celite, washed with dichloromethane (150 mL) and then with 5 wt % brine solution (400 g). The aqueous and organic layers were separated and the organic layer was concentrated to a dark oil (150 mL), which was used in the following step without purification.

### d. Preparation of 1-isopropyl-1H-indazole-3-carboxylic Acid {(1S,3R,5R)-8-(2,2-dihydroxyethyl)-8-aza-bicyclo[3.2.1]oct-3-yl}amide

A 1 L three-necked round bottom flask equipped with a magnetic stirrer, a nitrogen inlet and a thermometer was charged with the product of the previous step and water (250 mL) and heated to 50-55° C. To this solution was added 3N HCl (82 mL, 0.985 mol). The resulting mixture was stirred at 75° C. for 1 h. The reaction mixture was cooled to 25° C. and neutralized with 25 wt % NaOH (159 g, 0.99 mol) to pH 3.51. After about 20 min, the lower layer was collected (˜120 mL) to provide the title intermediate, which was used in the following step without purification.

### e. Synthesis of 1-isopropyl-1H-indazole-3-carboxylic acid {(1S,3R,5R)-8-[2-(4-acetylpiperazin-1-yl)ethyl]-8-azabicyclo[3.2.1]oct-3-yl}amide dihydrochloride

A 3 L three-necked round bottom flask equipped with a magnetic stirrer, a nitrogen inlet and a thermometer was charged with sodium triacetoxyborohydride (84 g, 0.349 mol) and dichloromethane (800 mL). The resulting mixture was stirred at 75° C. and charged with 1-acetylpiperazine (51 g, 0.394 mol). The addition assembly was rinsed with dichloromethane (30 mL). The mixture was stirred for 5 min and charged with the product of the previous step (˜120 mL) in 15 min maintaining the internal temperature less than 25° C. The mixture was stirred for 15 min, filtered through Celite and washed with dichloromethane (2×100 mL). The filtrate was washed with 1N NaOH (500 mL). The layers were separated and the lower organic layer was collected and concentrated to ˜150 mL.

Absolute ethanol (250 mL) was added and the mixture was concentrated to ˜200 mL. To this mixture, absolute ethanol (800 mL) was added and the mixture was heated to 40° C. To this mixture 3 N HCl (33 mL, 0.395 mol) was added in 3 min. The mixture was stirred for 10 min and crystallization began. The resulting suspension was stirred at 55° C. for 2 h and cooled to 25° C. The mixture was filtered though Whatman #2 filter paper and the wet cake was washed with absolute ethanol (2×100 mL). The product was dried under nitrogen for 30 min and then under vacuum at 40-50° C. for 24 h to provide the title compound (82 g).

### Example 1B

### Alternate Synthesis of 1-isopropyl-1H-indazole-3-carboxylic acid {(1S,3R,5R)-8-[2-(4-acetyl-piperazin-1-yl)-ethyl]-8-azabicyclo[3.2.1]oct-3-yl}amide dihydrochloride

### a. Preparation of (1R,5S)-8-[2-(4-acetylpiperazin-1-yl)ethyl]-8-azabicyclo[3.2.1]octan-3-one

Concentrated hydrochloric acid (50 mL) was added to a solution of 2,5-dimethoxy tetrahydrofuran (490.2 mL, 3.78 mol) in water (1200 mL). The resulting yellow solution was stirred at about 70-72° C. for about 2 h.

1-(2-Aminoethyl)piperazine (546.4 mL, 4.16 mol, 1.1 equiv.) was added to a solution of sodium acetate (1225.5 g) dissolved in water (3700 mL) at 15° C. Concentrated hydrochloric acid (350 mL) was added slowly keeping the internal temperature below 25° C. After cooling the mixture to 15° C., 3-oxopentanedioic acid (607.8 g, 4.16 mol) was added, and the solution temperature again cooled to 15° C. The aqueous yellow solution prepared above was added slowly over about 10 min, and the resulting yellow mixture was stirred at about 20° C. for about 30 min until carbon dixoide evolution slowed. The mixture was stirred at 40-45° C. for 2 h and the color of the reaction mixture turned dark brown.

The mixture was cooled to about 15° C. Aqueous sodium hydroxide (50%, ˜470 mL) was added in portions, keeping the temperature below 250, until pH 13 was reached. Sodium chloride (600 g) was added and the mixture was stirred to complete dissolution. The product was extracted with dichloromethane (1×2000 mL, 2×1500 mL). The combined organic phases were dried, filtered and the solution concentrated to 2500 mL.

The concentrated solution was cooled to 15° C. and acetic anhydride (500 mL) was added slowly, keeping the temperature below 25° C. The solution was stirred for 30 min and water (1500 mL) was added at 15° C. The mixture was stirred for 10 min, then acidified to pH 1 using 1M hydrochloric acid.

The DCM and aqueous phases were separated. Gas chromatography analysis revealed that there was no remaining product in the DCM phase. The aqueous phase was basefied to pH 14 by the portionwise addition of aqueous phase sodium hydroxide (50% in water, about 500 mL), keeping the internal temperature below 25° C. The product was extracted with DCM (3×1500 mL), and the collected organic phases (dark brown) were combined, dried, filtered through celite, and distilled to produce the title crude intermediate as a viscous brown oil (650 g, 94% purity). ¹H NMR (CDCl₃, 300 MHz) δ ppm: 3.65 (t, 2H), 3.56 (m, 2H), 3.48 (t, 2H), 2.78-2.47 (m, 10H), 2.23-2.02 (m, 4H), 2.10 (m, 3H), 1.61(m, 2H); ¹³C NMR (CDCl₃, 75 MHz) δ ppm: 209.81, 168.90, 59.10, 57.83, 53.83, 53.22, 48.01, 47.40, 46.19, 41.30, 27.80, 21.28.

### b. Preparation of (1R,5S)-8-[2-(4-acetylpiperazin-1-yl)ethyl]-8-azabicyclo[3.2.1]octan-3-one 1,5-naphthalene-disulfonic salt

The crude product of the previous step, (31.0 g, 0.111 mol) was dissolved in isopropyl alcohol (100 mL) at room temperature. The solution was heated to about 60° C. and 1,5-naphthalenedisulfonic acid tetrahydrate dissolved in isopropyl alcohol (70 mL) was added slowly over 1 h with stirring. After completion of the addition of the acid, the addition funnel was washed with isopropyl alcohol (50 mL). The mixture was stirred at about 60° C. for 1 h, cooled to room temperature, then stirred for 15 h. The mixture was filtered, and the resulting cake was washed with isopropyl alcohol (2×50 mL) and kept on the filter for 30 min. The product was then transferred to a flask and dried under high vacuum for 24 h to produce the title salt as a beige crystalline nonhydroscopic material (51.7 g). ¹H NMR (D₂O, 300 MHz) δ ppm: 8.84 (d, 2H), 8.20 (d, 2H), 7.73 (t, 2H), 4.23 (br s, 2H), 3.74 (m, 4H), 3.62 (s, 4H), 3.32 (m, 4H), 3.32-2.98 (m, 2H), 2.58 (d, 2H), 2.27 (m, 2H), 2.07 (s, 3H), 1.97 (d, 2H).

### c. Preparation of (1R,5S)-8-[2-(4-acetylpiperazin-1-yl)ethyl]-8-azabicyclo[3.2.1]octan-3-one (Free Base)

A stirred solution of (1R,5S)-8-[2-(4-acetylpiperazin-1-yl)ethyl]-8-azabicyclo-[3.2.1]-octan-3-one 1,5-naphthalene-disulfonic salt (51.6 g) in water (500 mL), cooled to 5° C., was basified to pH 14 using aqueous sodium hydroxide (50%), keeping the temperature below 15° C. DCM (300 mL) was added and the emulsion formed was filtered through celite. The layers were separated and the aqueous layer was washed with DCM (3×100 mL). The combined organic phases were dried for 24 h to produce the title intermediate (23.3 g, 93% yield based on the salt).

### d. Preparation of 1-{4-[2-((1R,3R,5S)-3-amino-8-azabicyclo[3.2.1]oct-8-yl)ethyl]piperazin-1-yl}ethanone

(1R,5S)-8-[2-(4-Acetylpiperazin-1-yl)ethyl]-8-azabicyclo[3.2.1]octan-3-one (58.2 g, 0.209 mol), prepared as described in the previous step, was dissolved in methanol (300 mL) at room temperature. To this solution, ammonium formate (63.1 g, 4.17 mol, 20 equiv.) was added, followed by the addition of methanol (100 mL) and water (85 mL). Palladium (wetted 10% palladium on activated carbon, 58 g) was added to the mixture, followed by the addition of methanol (150 mL). The reaction mixture was stirred at room temperature for about 20 h, until gas chromatography analysis revealed complete conversion of the ketone. The reaction mixture was filtered through celite, the resulting cake was washed with methanol (about 700 mL), and the solvent removed. The residue was dissolved in water (300 mL), the solution was cooled to 5° C., and the solution basified to pH 14 using 50% sodium hydroxide. The solution was saturated with sodium chloride and the product was extracted with DCM (400 mL). The aqueous phase was washed with DCM (3×150 mL). The combined organic fractions were dried for about 24 h under high vacuum to produce the title intermediate as a viscous light brown oil (43 g, 91% purity). ¹H NMR (CDCl₃, 300 MHz) δ ppm: 3.61 (t, 2H), 3.46 (t, 2H), 3.23 (t, 1H), 3.18 (m, 2H), 2.47 (m, 8H), 2.14-1.93 (m, 6H), 2.08 (s, 3H), 1.41 (d, 2H), 1.40 (br s, 2H); ¹³C NMR (CDCl₃, 75 MHz) δ ppm: 168.79, 58.87, 57.36, 53.69, 53.35, 53.12, 49.41, 46.13, 42.65, 41.22, 38.47, 26.39, 21.22.

### e. Preparation of 1-isopropyl-1H-indazole-3-carboxylic Acid {(1S,3R,5R)-8-[2-(4-acetyl-piperazin-1-yl)-ethyl]-8-azabicyclo[3.2.1]oct-3-yl}amide (Free Base)

1-Isopropyl-1H-indazole-3-carboxylic acid (21.16 g, 0.104 mol) was suspended in acetonitrile (150 mL) at room temperature. The mixture was cooled to 5° C. and triethylamine (17.4 mL, 0.124 mol, 1.2 equiv.) was added, resulting in a clear yellow solution. The mixture was stirred for 10 min and a solution of 1-{4-[2-((1R,3R,5S)-3-amino-8-azabicyclo[3.2.1]oct-8-yl)ethyl]piperazin-1-yl}ethanone (29.0 g, 0.104 mol) in acetonitrile (150 mL) was added in portions, keeping the temperature below 10° C. After 10 min stirring, PyBOP (54.97 g, 0.106 mol, 1.02 equiv.) dissolved in acetonitrile (150 mL) was added in portions, keeping the internal temperature below 15° C. The clear light brown solution was stirred at 5° C. for 30 min, slowly warmed-up and stirred at room temperature for about 2 h, at which point gas chromatography analysis showed complete conversion. The reaction mixture was diluted with isopropyl acetate (350 mL), and the product extracted with 1M hydrochloric acid (400 mL). After separation of the two layers, the organic layer was washed with 1M hydrochloric acid (3×100 mL). Analysis was carried out to confirm the full extraction of the product (less than 2% remaining). The combined aqueous phase was cooled to 5° C. with an ice bath and basified to pH 13 using 50% aqueous sodium hydroxide. After saturation with sodium chloride, the product was extracted with DCM (500 mL). The aqueous phase was washed with DCM (2×100 mL). The combined organic phases were dried under high vacuum for 24 h to produce the title compound (41.5 g, 92% purity).

### f. Synthesis of 1-isopropyl-1H-indazole-3-carboxylic Acid {(1S,3R,5R)-8-[2-(4-acetyl-piperazin-1-yl)-ethyl]-8-azabicyclo[3.2.1]oct-3-yl}amide dihydrochloride

1-Isopropyl-1H-indazole-3-carboxylic acid {(1S,3R,5R)-8-[2-(4-acetyl-piperazin-1-yl)-ethyl]-8-azabicyclo[3.2.1]oct-3-yl}amide free base was dissolved in ethanol (240 mL) at 35° C. The mixture was warmed to 35-40° C. (internal) and concentrated hydrochloric acid (14.3 mL, 2 equiv.) was added dropwise in a rate to keep the internal temperature between 35 and 45° C. After completion of the hydrochloric acid addition, seed crystals from an earlier small-scale synthesis prepared as described in Example 1A were added and precipitation occurred. The mixture was stirred at 35-40° C. for 1 h, slowly cooled down then stirred for another 1 h at room temperature, and the salt isolated by N₂ pressure filtration. The resulting cake was washed with ethanol (3×20 mL) and dried under high vacuum for 24 h to produce the title compound as a white solid (31.0 g, 70% yield). HPLC analysis gave a purity of 99.2%, the exo-isomer being less than 0.1%. ¹H NMR (D₂O, 300 MHz) δ ppm: 7.86 (d, 1H), 7.46 (d, 1H), 7.30 (t, 1H), 7.15 (t, 1H), 4.80 (sep, 1H), 4.13 (m, 3H), 3.86 (m, 4H), 3.70 (m, 2H), 3.57-3.40 (m, 6H), 2.55-2.49 (m, 2H), 2.42-2.28 (m, 6H), 2.12 (s, 3H), 1.42 (d, 6H).

### Example 2

### Synthesis of 1-isopropyl-1H-indazole-3-carboxylic Acid {(1S,3R,5R)-8-[2-(4-acetylpiperazin-1-yl)ethyl]-8-azabicyclo[3.2.1]oct-3-yl}amide dihydrobromide

A 500 mL three-necked round bottom flask equipped with a magnetic stirrer, a nitrogen inlet and a thermometer was charged with water (120 mL) and 1-isopropyl-1H-indazole-3-carboxylic acid {(1S,3R,5R)-8-[2-(4-acetylpiperazin-1-yl)ethyl]-8-azabicyclo[3.2.1]oct-3-yl}amide dihydrochloride (12 g, 22.2 mmol) prepared as in Example 1A. The resulting mixture was stirred to give a light yellow clear solution. To this solution was added 25 wt % NaOH (7.83 g, 24.4 mmol) in 2 min to give a white milky suspension. Dichloromethane (120 mL) was added and the mixture was stirred for 30 min to give a clear two-layer solution. The layers were separated to give an aqueous layer (113 mL) and an organic layer (125 mL), which was washed with 10% aqueous NaBr (120 mL). The layers were separated to give an organic layer (120 mL) which was concentrated to about one-quarter volume. Absolute ethanol (250 mL) was added and the mixture was distilled to give ˜200 mL total volume. The solution was stirred at 58° C. and 48 wt % aqueous HBr (8.2 g, 49 mmol) was added in 2 min. Precipitation was observed when more than half of the HBr was added. The mixture was stirred at 55° C. to 62° C. for 1 h and then cooled to ambient temperature and filtered. The filtrate was washed with absolute ethanol (40 mL), dried under nitrogen for 20 min and dried at 45° C. under vacuum for 48 h to give the title compound (13.42 g) as a white solid.

### Examples 3-8

### Properties of Salts of the Invention

Samples of the crystalline dihydrochloride salt of 1-isopropyl-1H-indazole-3-carboxylic acid {(1S,3R,5R)-8-[2-(4-acetylpiperazin-1-yl)ethyl]-8-azabicyclo[3.2.1]oct-3-yl}amide, prepared as in Example 1 and of the crystalline dihydrobromide salt of 1-isopropyl-1H-indazole-3-carboxylic acid {(1S,3R,5R)-8-[2-(4-acetylpiperazin-1-yl)ethyl]-8-azabicyclo[3.2.1]oct-3-yl}amide, prepared as in Example 2, were analyzed by powder x-ray diffraction (PXRD), differential scanning calorimetry (DSC), thermogravimetric analysis (TGA) infrared spectroscopy (IR) and elemental analysis.

### Example 3

### Powder X-Ray Diffraction

Powder x-ray diffraction patterns were obtained with a Thermo ARL X-Ray Diffractometer Model X'TRA (Thermo ARL SA, Switzerland) using Cu Kα radiation at 1.542 Å (45 kV, 40 mA) with a Si(Li) solid-state detector. The analysis was typically performed at a scan rate of 2′/min with a step size of 0.03° per point over a range of 2 to 30° in two-theta angle. Samples, either as received or ground to a fine powder, were gently packed into a custom small-volume insert designed to fit into the instrument top-loading sample cup for analysis. The instrument calibration to within ±0.02° two-theta angle was verified weekly by comparison with a silicon metal standard. Representative PXRD patterns for samples of the crystalline dihydrochloride, dihydrobromide, and of the hydrate of the dihydrochloride salts of the invention are shown in FIGS. 1, 3, and 7, respectively.

### Example 4

### Thermal Analysis

Differential scanning calorimetry (DSC) was performed using a TA Instruments

Model Q-100 module. Data were collected and analyzed using TA Instruments Thermal Advantage for Q Series™ software. A sample of about 1-2 mg was accurately weighed into an aluminum pan with lid. The sample was evaluated using a linear heating ramp of 5° C./min from ambient temperature to at least 225° C. The DSC cell was purged with dry nitrogen during use. Representative DSC traces for samples of the crystalline dihydrochloride and dihydrobromide salts of the invention are shown in FIGS. 2 and 4, respectively.

Thermogravimetric analysis (TGA) was performed using a TA Instruments Model Q-500 module. Data were collected and analyzed using TA Instruments Thermal Advantage for Q Series™ software. A sample weighing about 1-2 mg was placed in an aluminum pan on a platinum cradle and scanned from ambient temperature to 275° C. with a linear heating rate of either 2° C./min or 5° C./min. The balance and furnace chambers were purged with nitrogen during use. Representative TGA traces for samples of crystalline dihydrochloride and dihydrobromide salts of the invention are also shown in FIGS. 2 and 4, respectively.

The DSC trace in FIG. 2 demonstrates that a dihydrochloride salt of the present invention has excellent thermal stability with maximum in endothermic heat flow in the range of about 230° C. to about 275° C. and no significant thermal events below about 200° C. Comparison of the DSC and TGA traces indicates that a dihydrochloride salt of the present invention undergoes simultaneous melting and decomposition at temperatures above about 180-200° C.

### Example 5

### Dynamic Moisture Sorption Assessment

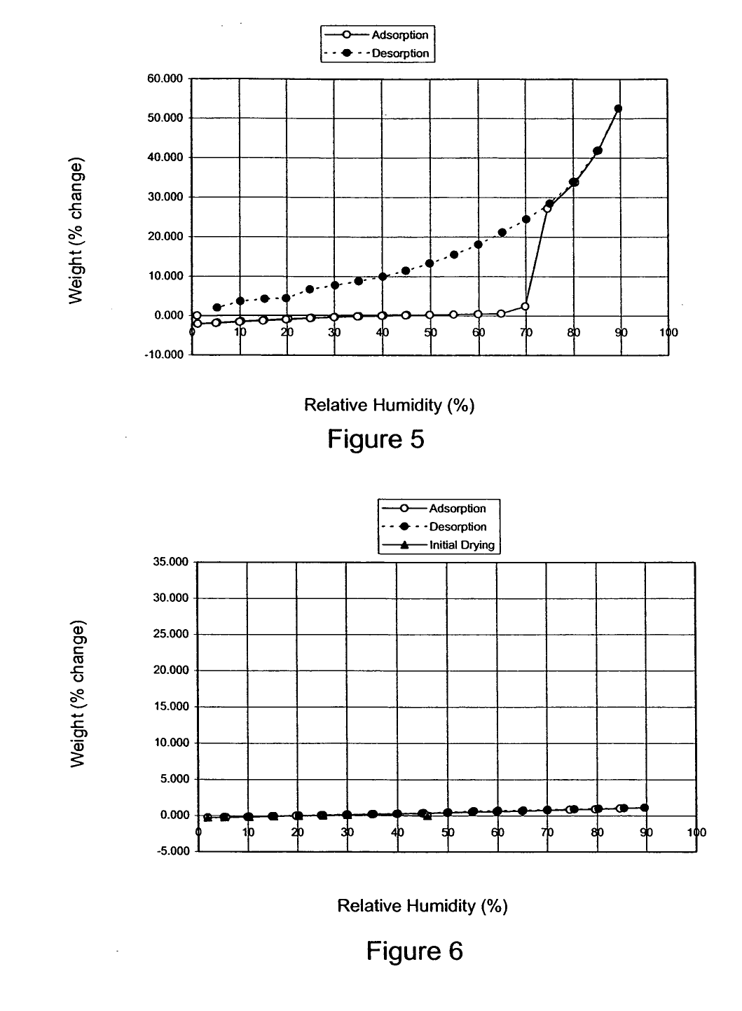

Dynamic moisture sorption (DMS) assessment was performed at 25° C. using a VTI atmospheric microbalance, SGA-100 system (VTI Corp., Hialeah, Fla. 33016). A sample size of approximately 5-10 mg was used and the humidity was set at the ambient value at the start of the analysis. A typical DMS analysis consisted of three scans: ambient to 2% relative humidity (RH), 2% RH to 90% RH, 90% RH to 5% RH at a scan rate of 5% RH/step. The mass was measured every two minutes and the RH was changed to the next value (+5% RH) when the mass of the sample was stable to within 0.01% for 5 consecutive points. Representative DMS traces for samples of crystalline dihydrochloride and dihydrobromide salts of the invention are shown in FIGS. 5 and 6, respectively.

### Example 6

### Infrared Analysis

The infrared (IR) absorption spectrum was determined over the frequency range 4000 to 675 cm⁻¹ using an Avatar 360 FT-IR spectrometer equipped with a Nicolet attenuated total reflection (ATR) sample holder. A representative IR absorption spectrum for a sample of a crystalline dihydrochloride salt of the invention had significant absorption bands at 753±1, 986±1, 1086±1, 1197±1, 1267±1, 1281±1, 1437±1, 1488±1, 1522±1, 1638±1, 1664±1, and 2416±cm⁻¹.

### Example 7

### Solid State Stability Assessment

Samples of the dihydrochloride salt of the invention, about 100 mg each, were stored in multiple 4 mL glass vials with Teflon lined lids at 25° C. and 60% relative humidity (RH) and at 40° C. and 75% RH in open and closed containers. At specific intervals, the contents of a representative vial was removed and analyzed by the Karl Fisher method for water content and by the following HPLC method:
Column: Agilent Zorbax Bonus RP, 4.6×250 mm, 5 μm (Agilent Part No. 880668-901);
Mobile Phase A: 88% water, 2% acetonitrile, 10% 100 mM potassium phosphate;
Mobile Phase B: 20% water, 70% acetonitrile; 10% 100 mM potassium phosphate;
Flow rate: 1.5 mL/min;
Injection Volume: 10 μL
Detector: 214 nm;
Gradient—Time in minutes (% Mobile Phase B): 0.0 (2); 1.0 (2); 9.0 (22);
19.0 (28); 33.0 (63); 36.3 (100); 37.0 (2); and 42.0 (2).

Samples were prepared as 0.25 to 0.75 mg/mL solutions in 5% acetonitrile, 85% water, 10% 100 mM potassium phosphate for injection onto the HPLC.

The initial potency of the samples as compared with the freebase compound of formula I was 82.4% as determined by HPLC area percentage. After 6 months of storage, for the samples kept under all conditions, there was no detectable change in potency. The initial water content was 1.2%, as determined by Karl Fischer titration using a Brinkman Metrohm (Westbury, N.Y.) Karl Fischer Model 831 coulometer. After 6 months the water content of the samples stored in open containers at 25° C./60% RH and at 40° C./75% RH was 2.9% and 5.3%, respectively, an increase of 1.7% and 4.1%, respectively.

### Example 8

### Determination of Counterion Molar Ratio

The counterion molar ratio of halide acid (HX) to 1-isopropyl-1H-indazole-3-carboxylic acid {(1S,3R,5R)-8-[2-(4-acetylpiperazin-1-yl)ethyl]-8-azabicyclo[3.2.1]oct-3-yl}amide (compound of formula I) was calculated according to the following formula:
Counterion Ratio=(W_{HX}/MW_{HX})/(W_{I}/MW_{I})
where W_{HX} is the weight percentage of HX in the sample, MW_{HX} is the molecular weight of HX, MW_{I} is the molecular weight of compound of formula I (466.6 amu), and W_{I} is the weight percentage of compound of formula I in the sample, calculated according to the formula:
W_{I}=100−W_{HX}−W_{H2O}−W_{RS}
where W_{H2O} is the weight percentage water content, W_{RS} is the weight percentage residual solvent, under the assumption that compound I has no impurities.

The molar ratio of hydrochloric acid to compound I for one sample of a crystalline salt of the invention and of hydrobromic acid to compound I for two samples of a crystalline salt of the invention is given below in Table I. The weight percentage of HCl or HBr (W_{HX}) was determined by titration, the water content W_{H2O} was determined by coulometric Karl Fischer titration and the residual solvent content W_{RS} was determined by gas chromatography.

TABLE I
Hydrochloride and Hydrobromide
Salts of Compound of Formula I

| | Hydrochloride | Hydrobromide | Hydrobromide |
|---|---|---|---|
| | salt | salt | salt |
| MW_{I} | 466.62 | 466.62 | 466.62 |
| MW_{HX} | 36.46 | 80.91 | 80.91 |
| W_{HX} | 13 | 24.36 | 23.87 |
| W_{H2O} | 2.1 | 0.8 | 0.82 |
| W_{RS} | 0 | 1.91 | 2.09 |
| Counterion ratio | 1.96 | 1.93 | 1.88 |

### Example 9

### Crystalline hydrate of 1-isopropyl-1H-indazole-3-carboxylic Acid {(1S,3R,5R)-8-[2-(4-acetylpiperazin-1-yl)ethyl]-8-azabicyclo[3.2.1]oct-3-yl}amide dihydrochloride

A sample of the dihydrochloride salt of the invention, about 50 mg, was stored in a 20 mL glass vial at 40° C. and 75% relative humidity. At specific intervals, aliquots of sample were dispensed and analyzed by PXRD and by DSC and TGA. Analysis results for a sample stored for eight weeks are shown in FIGS. 7 and 8, respectively. The low temperature (i.e. below 150° C.) features in the DSC and TGA traces are characteristic of a hydrate. The hydrate form is observed to be stable when stored at 40° C. and 75% RH. Comparison of the PXRD spectra of a sample stored for 8 weeks at 40° C./75% RH and of a sample stored at 12 weeks at 40° C./75% RH showed no significant change in PXRD peak positions.

### Comparative Example 1

### Synthesis of fumaric Acid Salt of 1-isopropyl-1H-indazole-3-carboxylic acid {(1S,3R,5R)-8-[2-(4-acetylpiperazin-1-yl)ethyl]-8-azabicyclo[3.2.1]oct-3-yl}amide

To a solution of 1-isopropyl-1H-indazole-3-carboxylic acid {(1S,3R,5R)-8-[2-(4-acetylpiperazin-1-yl)ethyl]-8-azabicyclo[3.2.1]oct-3-yl}amide (0.1 g, 0.21 mmol) in 50% acetonitrile/water (1 mL) was added an ethanolic 1M fumaric acid solution (0.44 mL 0.42 mmol). The resulting solution was lyophilized overnight and then mixed with ethyl acetate (1 mL). Hot ethanol was added to this mixture with heating until a homogeneous solution was obtained (0.4 mL). The resulting clear solution was then allowed to crystallize at room temperature. The resulting solid was filtered, washed with ethanol, and dried under vacuum to give the title compound as a crystalline solid (0.13 g).

### Comparative Example 2

### Synthesis of Phosphoric Acid Salt of 1-isopropyl-1H-indazole-3-carboxylic Acid {(1S,3R,5R)-8-[2-(4-acetylpiperazin-1-yl)ethyl]-8-azabicyclo[3.2.1]oct-3-yl}amide

To a solution of 1-isopropyl-1H-indazole-3-carboxylic acid {(1S,3R,5R)-8-[2-(4-acetylpiperazin-1-yl)ethyl]-8-azabicyclo[3.2.1]oct-3-yl}amide (0.2 g, 0.43 mmol) in methanol (3 mL) was added an ethanolic 1M phosphoric acid solution (0.43 mL, 0.43 mmol). The resulting heterogeneous solution was then heated to solubilize, filtered, and allowed to cool overnight. The resulting solid was filtered, washed with methanol and dried under vacuum to give the title compound as a crystalline solid (0.08 g).

### Comparative Example 3

### Synthesis of p-toluene Sulfonic Acid Salt of 1-isopropyl-1H-indazole-3-carboxylic acid {(1S,3R,5R)-8-[2-(4-acetylpiperazin-1-yl)ethyl]-8-azabicyclo[3.2.1]oct-3-yl}amide

A solution of 1-isopropyl-1H-indazole-3-carboxylic acid {(1S,3R,5R)-8-[2-(4-acetylpiperazin-1-yl)ethyl]-8-azabicyclo[3.2.1]oct-3-yl}amide (38.7 mg, 0.08 mmol) in isopropanol (2 mL) was heated in a 75° C. water bath and solid p-toluenesulfonic acid monohydrate (32.3 mg, 0.17 mmol) was added. The resulting solution was heated until the solids dissolved and then allowed to cool to room temperature. Crystals of the title compound formed overnight.

### Comparative Example 4

### Synthesis of Acid Salts of 1-isopropyl-1H-indazole-3-carboxylic Acid {(1S,3R,5R)-8-[2-(4-acetylpiperazin-1-yl)ethyl]-8-azabicyclo[3.2.1]oct-3-yl}amide

Using procedures similar to those of Comparative Examples 1-3, the following acid salts of 1-isopropyl-1H-indazole-3-carboxylic acid {(1S,3R,5R)-8-[2-(4 acetylpiperazin-1-yl)ethyl]-8-azabicyclo[3.2.1]oct-3-yl}amide were prepared in solid form using the number of equivalents of acid indicated in parentheses: acetate (2); benzoate (2); nitrate (2); propionate (1); tartrate (2); phosphate (0.5).

### Comparative Example 5

### Properties of Compounds of Comparative Examples 1-4

The crystalline acid salts of the compound of formula I of Comparative Examples 1-4 were analyzed by PXRD, DSC, and TGA. The temperatures at which endothermic heat flow was observed by DSC, and weight loss was observed by TGA, along with confirmation of crystallinity by PXRD are summarized in Table II where the stoichiometry is indicated by the number of molar equivalents of acid used to prepare the acid salt.

TABLE II
Comparison Salts

| | Equivalents | DSC | | |
|---|---|---|---|---|
| Salt | of acid | endotherm | TGA | PXRD |
| acetate | 2 | ˜100° C. | wt. loss | crystalline |
| | | | ˜100° C. | |
| benzoate | 2 | <150° C. | wt. loss | crystalline |
| | | | maximum | |
| | | | ˜100° C. | |
| fumarate | 2 | <150° C. | 5-6% wt. | crystalline |
| | | | loss ˜100° C. | |
| nitrate | 2 | <100° C. | 5-6% wt. | crystalline |
| | | | loss ˜150° C. | |
| phosphate | 0.5 | <100° C., | 3-5% wt. | crystalline |
| | | >200° C. | loss <100° C. | |
| phosphate | 1 | <100° C., | 3-5% wt. | crystalline |
| | | >200° C. | loss <100° C. | |
| propionate | 1 | several | wt. loss | crystalline |
| | | ˜100° C. | <100° C. | |
| tartrate | 2 | <100° C., | wt. loss | crystalline |
| | | ˜160° C. | <100° C. | |
| tosylate | 2 | >200° C. | | crystalline* |

*multiple polymorphs

Assay 1: Radioligand Binding Assay on 5-HT_{4(c)} Human Receptors

a. Membrane Preparation 5-HT_{4(c)}

HEK-293 (human embryonic kidney) cells stably-transfected with human 5-HT_{4(c)} receptor cDNA (Bmax=˜6.0 pmol/mg protein, as determined using [³H]-GR113808 membrane radioligand binding assay) were grown in T-225 flasks in Dulbecco's Modified Eagles Medium (DMEM) containing 4,500 mg/L D-glucose and pyridoxine hydrochloride (GIBCO-Invitrogen Corp., Carlsbad Calif.: Cat #11965) supplemented with 10% fetal bovine serum (FBS) (GIBCO-Invitrogen Corp.: Cat #10437), 2 mM L-glutamine and (100 units) penicillin-(100 μg) streptomycin/ml (GIBCO-Invitrogen Corp.: Cat #15140) in a 5% CO₂, humidified incubator at 37° C. Cells were grown under continuous selection pressure by the addition of 800 μg/mL geneticin (GIBCO-Invitrogen Corp.: Cat #10131) to the medium.

Cells were grown to roughly 60-80% confluency (<35 subculture passages). At 20-22 hours prior to harvesting, cells were washed twice and fed with serum-free DMEM. All steps of the membrane preparation were performed on ice. The cell monolayer was lifted by gentle mechanical agitation and trituration with a 25 mL pipette. Cells were collected by centrifugation at 1000 rpm (5 min).

For the membrane preparation, cell pellets were resuspended in ice-cold 50 mM 4-(2-hydroxyethyl)-1-piperazineethanesulphonic acid (HEPES), pH 7.4 (membrane preparation buffer) (40 mL/total cell yield from 30-40 T225 flasks) and homogenized using a polytron disrupter (setting 19, 2×10 s) on ice. The resultant homogenates were centrifuged at 1200 g for 5 min at 4° C. The pellet was discarded and the supernatant centrifuged at 40,000 g (20 min). The pellet was washed once by resuspension with membrane preparation buffer and centrifugation at 40,000 g (20 min). The final pellet was resuspended in 50 mM HEPES, pH 7.4 (assay buffer) (equivalent 1 T225 flask/1 mL). Protein concentration of the membrane suspension was determined by the method of Bradford (Bradford, 1976). Membranes were stored frozen in aliquots at −80° C.

b. Radioligand Binding Assays

Radioligand binding assays were performed in 1.1 mL 96-deep well polypropylene assay plates (Axygen) in a total assay volume of 400 μL containing 2 μg membrane protein in 50 mM HEPES pH 7.4, containing 0.025% bovine serum albumin (BSA). Saturation binding studies for determination of K_{d} values of the radioligand were performed using [³H]-GR113808 (Amersham Inc., Bucks, UK: Cat #TRK944; specific activity ˜82 Ci/mmol) at 8-12 different concentrations ranging from 0.001 nM-5.0 nM. Displacement assays for determination of pKᵢ values of compounds were performed with [³H]-GR113808 at 0.15 nM and eleven different concentrations of compound ranging from 10 pM-100 μM.

Test compounds were received as 10 mM stock solutions in DMSO and diluted to 400 μM into 50 mM HEPES pH 7.4 at 25° C., containing 0.1% BSA, and serial dilutions (1:5) then made in the same buffer. Non-specific binding was determined in the presence of 1 μM unlabeled GR113808. Assays were incubated for 60 min at room temperature, and then the binding reactions were terminated by rapid filtration over 96-well GF/B glass fiber filter plates (Packard BioScience Co., Meriden, Conn.) presoaked in 0.3% polyethyleneimine. Filter plates were washed three times with filtration buffer (ice-cold 50 mM HEPES, pH7.4) to remove unbound radioactivity. Plates were dried, 35 μL Microscint-20 liquid scintillation fluid (Packard BioScience Co., Meriden, Conn.) was added to each well and plates were counted in a Packard Topcount liquid scintillation counter (Packard BioScience Co., Meriden, Conn.).

Binding data were analyzed by nonlinear regression analysis with the GraphPad Prism Software package (GraphPad Software, Inc., San Diego, Calif.) using the 3-parameter model for one-site competition. The BOTTOM (curve minimum) was fixed to the value for nonspecific binding, as determined in the presence of 1 μM GR113808. Kᵢ values for test compounds were calculated, in Prism, from the best-fit IC₅₀ values, and the K_{d} value of the radioligand, using the Cheng-Prusoff equation (Cheng and Prusoff, Biochemical Pharmacology, 1973, 22, 3099-108): Kᵢ=IC₅₀/(1+[L]/K_{d}) where [L]=concentration [³H]-GR113808. Results are expressed as the negative decadic logarithm of the Kᵢ values, pKᵢ.

Test compounds having a higher pKᵢ value in this assay have a higher binding affinity for the 5-HT₄ receptor. The compound of formula I had a pKᵢ value greater than about 7 in this assay.

Assay 2: Radioligand Binding Assay on 5-HT_{3A} Human Receptors: Determination of Receptor Subtype Selectivity

a. Membrane Preparation 5-HT_{3A}

HEK-293 (human embryonic kidney) cells stably-transfected with human 5-HT_{3A} receptor cDNA were obtained from Dr. Michael Bruess (University of Bonn, GDR) (Bmax=˜9.0 pmol/mg protein, as determined using [³H]-GR65630 membrane radioligand binding assay). Cells were grown in T-225 flasks or cell factories in 50% Dulbecco's Modified Eagles Medium (DMEM) (GIBCO-Invitrogen Corp., Carlsbad, Calif.: Cat #11965) and 50% Ham's F12 (GIBCO-Invitrogen Corp.: Cat #11765) supplemented with 10% heat inactivated fetal bovine serum (FBS) (Hyclone, Logan, Utah: Cat #SH30070.03) and (50 units) penicillin-(50 μg) streptomycin/ml (GIBCO-Invitrogen Corp.: Cat #15140) in a 5% CO₂, humidified incubator at 37° C.

Cells were grown to roughly 70-80% confluency (<35 subculture passages). All steps of the membrane preparation were performed on ice. To harvest the cells, the media was aspirated and cells were rinsed with Ca²⁺, Mg²⁺-free Dulbecco's phosphate buffered saline (dPBS). The cell monolayer was lifted by gentle mechanical agitation. Cells were collected by centrifugation at 1000 rpm (5 min). Subsequent steps of the membrane preparation followed the protocol described above for the membranes expressing 5-HT_{4(c)} receptors.

b. Radioligand Binding Assays

Radioligand binding assays were performed in 96-well polypropylene assay plates in a total assay volume of 200 μL containing 1.5-2 μg membrane protein in 50 mM HEPES pH 7.4, containing 0.025% BSA assay buffer. Saturation binding studies for determination of K_{d} values of the radioligand were performed using [³H]-GR65630 (PerkinElmer Life Sciences Inc., Boston, Mass.: Cat #NET1011, specific activity ˜85 Ci/mmol) at twelve different concentrations ranging from 0.005 nM to 20 nM. Displacement assays for determination of pKᵢ values of compounds were performed with [³H]-GR65630 at 0.50 nM and eleven different concentrations of compound ranging from 10 pM to 100 μM. Compounds were received as 10 mM stock solutions in DMSO (see section 3.1), diluted to 400 μM into 50 mM HEPES pH 7.4 at 25° C., containing 0.1% BSA, and serial (1:5) dilutions then made in the same buffer. Non-specific binding was determined in the presence of 10 μM unlabeled MDL72222. Assays were incubated for 60 min at room temperature, then the binding reactions were terminated by rapid filtration over 96-well GF/B glass fiber filter plates (Packard BioScience Co., Meriden, Conn.) presoaked in 0.3% polyethyleneimine. Filter plates were washed three times with filtration buffer (ice-cold 50 mM HEPES, pH7.4) to remove unbound radioactivity. Plates were dried, 35 μL Microscint-20 liquid scintillation fluid (Packard BioScience Co., Meriden, Conn.) was added to each well and plates were counted in a Packard Topcount liquid scintillation counter (Packard BioScience Co., Meriden, Conn.).

Binding data were analyzed using the non-linear regression procedure described above to determine Kᵢ values. The BOTTOM (curve minimum) was fixed to the value for nonspecific binding, as determined in the presence of 10 μM MDL72222. The quantity [L] in the Cheng-Prusoff equation was defined as the concentration [³H]-GR65630.

Selectivity for the 5-HT₄ receptor subtype with respect to the 5-HT₃ receptor subtype was calculated as the ratio Kᵢ(5-HT_{3A})/Kᵢ(5-HT_{4(c)}). The compound of formula I had a 5-HT₄/5-HT₃ receptor subtype selectivity greater than about 500 in this assay.

Assay 3: Whole-cell cAMP Accumulation Flashplate Assay with HEK-293 cells expressing human 5-HT_{4(c)} Receptors

In this assay, the functional potency of a test compound was determined by measuring the amount of cyclic AMP produced when HEK-293 cells expressing 5-HT₄ receptors were contacted with different concentrations of test compound.

a. Cell Culture

HEK-293 (human embryonic kidney) cells stably-transfected with cloned human 5-HT_{4(c)} receptor cDNA were prepared expressing the receptor at two different densities: (1) at a density of about 0.5-0.6 pmol/mg protein, as determined using a [³H]-GR113808 membrane radioligand binding assay, and (2) at a density of about 6.0 pmol/mg protein. The cells were grown in T-225 flasks in Dulbecco's Modified Eagles Medium (DMEM) containing 4,500 mg/L D-glucose (GIBCO-Invitrogen Corp.: Cat #11965) supplemented with 10% fetal bovine serum (FBS) (GIBCO-Invitrogen Corp.: Cat #10437) and (100 units) penicillin-(100 μg) streptomycin/ml (GIBCO-Invitrogen Corp.: Cat #15140) in a 5% CO₂, humidified incubator at 37° C. Cells were grown under continuous selection pressure by the addition of geneticin (800 μg/mL: GIBCO-Invitrogen Corp.: Cat #10131) to the medium.

b. Cell Preparation

Cells were grown to roughly 60-80% confluency. Twenty to twenty-two hours prior to assay, cells were washed twice, and fed, with serum-free DMEM containing 4,500 mg/L D-glucose (GIBCO-Invitrogen Corp.: Cat #11965). To harvest the cells, the media was aspirated and 10 mL Versene (GIBCO-Invitrogen Corp.: Cat #15040) was added to each T-225 flask. Cells were incubated for 5 min at RT and then dislodged from the flask by mechanical agitation. The cell suspension was transferred to a centrifuge tube containing an equal volume of pre-warmed (37° C.) dPBS and centrifuged for 5 min at 1000 rpm. The supernatant was discarded and the pellet was re-suspended in pre-warmed (37° C.) stimulation buffer (10 mL equivalent per 2-3 T-225 flasks). This time was noted and marked as time zero. The cells were counted with a Coulter counter (count above 8 μm, flask yield was 1-2×10⁷ cells/flask). Cells were resuspended at a concentration of 5×10⁵ cells/ml in pre-warmed (37° C.) stimulation buffer (as provided in the flashplate kit) and preincubated at 37° C. for 10 min.

cAMP assays were performed in a radioimmunoassay format using the Flashplate Adenylyl Cyclase Activation Assay System with ¹²⁵I-cAMP (SMP004B, PerkinElmer Life Sciences Inc., Boston, Mass.), according to the manufacturer's instructions.

Cells were grown and prepared as described above. Final cell concentrations in the assay were 25×10³ cells/well and the final assay volume was 100 μL. Test compounds were received as 10 mM stock solutions in DMSO, diluted to 400 μM into 50 mM HEPES pH 7.4 at 25° C., containing 0.1% BSA, and serial (1:5) dilutions then made in the same buffer. Cyclic AMP accumulation assays were performed with 11 different concentrations of compound ranging from 10 pM to 100 μM (final assay concentrations). A 5-HT concentration-response curve (10 pM to 100 μM) was included on every plate. The cells were incubated, with shaking, at 37° C. for 15 min and the reaction terminated by addition of 100 μl of ice-cold detection buffer (as provided in the flashplate kit) to each well. The plates were sealed and incubated at 4° C. overnight. Bound radioactivity was quantified by scintillation proximity spectroscopy using the Topcount (Packard BioScience Co., Meriden, Conn.).

The amount of cAMP produced per mL of reaction was extrapolated from the cAMP standard curve, according to the instructions provided in the manufacturer's user manual. Data were analyzed by nonlinear regression analysis with the GraphPad Prism Software package using the 3-parameter sigmoidal dose-response model (slope constrained to unity). Potency data are reported as pEC₅₀ values, the negative decadic logarithm of the EC₅₀ value, where EC₅₀ is the effective concentration for a 50% maximal response.

Test compounds exhibiting a higher pEC₅₀ value in this assay have a higher potency for agonizing the 5-HT₄ receptor. The compound of formula I which was tested in this assay in the cell line (1) having a density of about 0.5-0.6 pmol/mg protein, had a pEC₅₀ value greater than about 7.5.

Assay 4: In Vitro Voltage Clamp Assay of Inhibition of Potassium Ion Current in Whole Cells Expressing the hERG Cardiac Potassium Channel

CHO-K1 cells stably transfected with hERG cDNA were obtained from Gail Robertson at the University of Wisconsin. Cells were held in cryogenic storage until needed. Cells were expanded and passaged in Dulbecco's Modified Eagles Medium/F 12 supplemented with 10% fetal bovine serum and 200 μg/mL geneticin. Cells were seeded onto poly-D-lysine (100 μg/mL) coated glass coverslips, in 35 mm² dishes (containing 2 mL medium) at a density that enabled isolated cells to be selected for whole cell voltage-clamp studies. The dishes were maintained in a humidified, 5% CO₂ environment at 37° C.

Extracellular solution was prepared at least every 7 days and stored at 4° C. when not in use. The extracellular solution contained (mM): NaCl (137), KCl (4), CaCl₂ (1.8), MgCl₂ (1), Glucose (10), 4-(2-hydroxyethyl)-1-piperazineethanesulphonic acid (HEPES) (10), pH 7.4 with NaOH. The extracellular solution, in the absence or presence of test compound, was contained in reservoirs, from which it flowed into the recording chamber at approximately 0.5 mL/min. The intracellular solution was prepared, aliquoted and stored at −20° C. until the day of use. The intracellular solution contained (mM): KCl (130), MgCl₂ (1), ethylene glycol-bis(beta-aminoethyl ether) N,N,N′,N′-tetra acetic acid salt (EGTA) (5), MgATP (5), 4-(2-hydroxyethyl)-1-piperazineethanesulphonic acid (HEPES) (10), pH 7.2 with KOH. All experiments were performed at room temperature (20-22° C.).

The coverslips on which the cells were seeded were transferred to a recording chamber and perfused continuously. Gigaohm seals were formed between the cell and the patch electrode. Once a stable patch was achieved, recording commenced in the voltage clamp mode, with the initial holding potential at −80 mV. After a stable whole-cell current was achieved, the cells were exposed to test compound. The standard voltage protocol was: step from the holding potential of −80 mV to +20 mV for 4.8 sec, repolarize to −50 mV for 5 sec and then return to the original holding potential (−80 mV). This voltage protocol was run once every 15 sec (0.067 Hz). Peak current amplitudes during the repolarization phase were determined using pClamp software. Test compounds at a concentration of 3 μM were perfused over the cells for 5 minutes, followed by a 5-minute washout period in the absence of compound. Finally a positive control (cisapride, 20 nM) was added to the perfusate to test the function of the cell. The step from −80 mV to +20 mV activates the hERG channel, resulting in an outward current. The step back to −50 mV results in an outward tail current, as the channel recovers from inactivation and deactivates.

Peak current amplitudes during the repolarization phase were determined using pCLAMP software. The control and test article data were exported to Origin® (OriginLab Corp., Northampton Mass.) where the individual current amplitudes were normalized to the initial current amplitude in the absence of compound. The normalized current means and standard errors for each condition were calculated and plotted versus the time course of the experiment.

Comparisons were made between the observed K⁺ current inhibitions after the five-minute exposure to either the test article or vehicle control (usually 0.3% DMSO). Statistical comparisons between experimental groups were performed using a two-population, independent t-test (Microcal Origin v. 6.0). Differences were considered significant at p<0.05.

The smaller the percentage inhibition of the potassium ion current in this assay, the smaller the potential for test compounds to change the pattern of cardiac repolarization when used as therapeutic agents. The compound of formula I was tested in this assay at a concentration of 3 μM and exhibited an inhibition of the potassium ion current of less than about 15%.

Assay 5: In Vitro Model of Oral Bioavailability: Caco-2 Permeation Assay

The Caco-2 permeation assay was performed to model the ability of test compounds to pass through the intestine and get into the blood stream after oral administration. The rate at which test compounds in solution permeate a cell monolayer designed to mimic the tight junction of human small intestinal monolayers was determined.

Caco-2 (colon, adenocarcinoma; human) cells were obtained from ATCC (American Type Culture Collection; Rockville, Md.). For the permeation study, cells were seeded at a density of 63,000 cells/cm² on pre-wetted transwells polycarbonate filters (Costar; Cambridge, Mass.). A cell monolayer was formed after 21 days in culture. Following cell culture in the transwell plate, the membrane containing the cell monolayer was detached from the transwell plate and inserted into the diffusion chamber (Costar; Cambridge, Mass.). The diffusion chamber was inserted into the heating block which was equipped with circulating external, thermostatically regulated 37° C. water for temperature control. The air manifold delivered 95% O₂/5% CO₂ to each half of a diffusion chamber and created a laminar flow pattern across the cell monolayer, which was effective in reducing the unstirred boundary layer.

The permeation study was performed with test compound concentrations at 100 μM and with ¹⁴C-mannitol to monitor the integrity of the monolayer. All experiments were conducted at 37° C. for 60 min. Samples were taken at 0, 30 and 60 min from both the donor and receiver sides of the chamber. Samples were analyzed by HPLC or liquid scintillation counting for test compound and mannitol concentrations. The permeation coefficient (Kₚ) in cm/sec was calculated.

In this assay, a Kₚ value greater than about 10×10⁻⁶ cm/sec is considered indicative of favorable bioavailability. The compound of formula I was tested in this assay and exhibited a Kₚ value greater than about 20×10⁻⁶ cm/sec.

Assay 6: Pharmacokinetic Study in the Rat

Aqueous solution formulations of test compounds were prepared in 0.1% lactic acid at a pH of between about 5 and about 6. Male Sprague-Dawley rats (CD strain, Charles River Laboratories, Wilmington, Mass.) were dosed with test compounds via intravenous administration (IV) at a dose of 2.5 mg/kg or by oral gavage (PO) at a dose of 5 mg/kg. The dosing volume was 1 mL/kg for IV and 2 mL/kg for PO administration. Serial blood samples were collected from animals pre-dose, and at 2 (IV only), 5, 15, and 30 min, and at 1, 2, 4, 8, and 24 hours post-dose. Concentrations of test compounds in blood plasma were determined by liquid chromatography-mass spectrometry analysis (LC-MS/MS) (MDS SCIEX, API 4000, Applied Biosystems, Foster City, Calif.) with a lower limit of quantitation of 1 ng/mL.

Standard pharmacokinetic parameters were assessed by non-compartmental analysis (Model 201 for IV and Model 200 for PO) using WinNonlin (Version 4.0.1, Pharsight, Mountain View, Calif.). The maximum in the curve of test compound concentration in blood plasma vs. time is denoted Cₘₐₓ. The area under the concentration vs. time curve from the time of dosing to the last measurable concentration (AUC(0-t)) was calculated by the linear trapezoidal rule. Oral bioavailability (F(%)), i.e. the dose-normalized ratio of AUC(0-t) for PO administration to AUC(0-t) for IV administration, was calculated as:
F(%)=AUC_{PO}/AUC_{IV}×Dose_{IV}/Dose_{PO}×100%

Test compounds which exhibit larger values of the parameters Cₘₐₓ, AUC(0-t), and F(%) in this assay are expected to have greater bioavailability when administered orally. The compound of formula I had a Cₘₐₓ value of 0.25 μg/mL, an AUC(0-t) value of 0.73 μg·hr/mL and oral bioavailability (F(%)) in the rat model of about 100%.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto. Additionally, all publications, patents, and patent documents cited hereinabove are incorporated by reference herein in full, as though individually incorporated by reference.

What is claimed is:

1. A crystalline halide salt of 1-isopropyl-1H-indazole-3-carboxylic acid {(1S,3R,5R)-8-[2-(4-acetylpiperazin-1-yl)ethyl]-8-azabicyclo[3.2.1]oct-3-yl}amide or a solvate thereof.

2. The compound of claim 1 wherein the halide salt is a dihydrochloride salt.

3. The compound of claim 2, wherein the compound is characterized by a powder x-ray diffraction pattern having two or more diffraction peaks at 2θ values selected from 4.17±0.20, 8.23±0.20, 12.30±0.20, 12.77±0.20, 13.94±0.20, 16.04±0.20, 17.06±0.20, 18.56±0.20, 19.93±0.20, 20.49±0.20, 21.72±0.20, 22.58±0.20, 26.27±0.20, and 26.59±0.20.

4. The compound of claim 3 wherein the powder x-ray diffraction pattern comprises two or more diffraction peaks at 2θ values selected from 4.17±0.20, 8.23±0.20, 12.30±0.20, 13.94±0.20, and 18.56±0.20.

5. The compound of claim 2, wherein the compound is characterized by an powder x-ray diffraction pattern in which the peak positions are substantially in accordance with the peak positions of the pattern shown in FIG. 1.

6. The compound of claim 2, wherein the compound is characterized by a differential scanning calorimetry trace which shows a maximum in endothermic heat flow at a temperature greater than about 230° C.

7. The compound of claim 2, wherein the compound is characterized by an differential scanning calorimetry trace substantially in accordance with that shown in FIG. 2.

8. The compound of claim 1 wherein the halide salt is a dihydrobromide salt.

9. The compound of claim 8, wherein the compound is characterized by a powder x-ray diffraction pattern having two or more diffraction peaks at 2θ values selected from 4.41±0.20, 7.63±0.20, 8.75±0.20, 13.11±0.20, 14.52±0.20, 15.28±0.20, 21.89±0.20, 22.75±0.20, 23.35±0.20, 25.40±0.20, 26.34±0.20, 28.53±0.20, and 28.86±0.20.

10. The compound of claim 9 wherein the powder x-ray diffraction pattern comprises two or more diffraction peaks at 2θ values selected from 4.41±0.20, 8.75±0.20, 13.11±0.20, and 21.89±0.20.

11. The compound of claim 8, wherein the compound is characterized by an powder x-ray diffraction pattern in which the peak positions are substantially in accordance with the peak positions of the pattern shown in FIG. 3.

12. The compound of claim 1 wherein the compound is a hydrate of a dihydrochloride salt.

13. The compound of claim 12 wherein the compound is characterized by a powder x-ray diffraction pattern having two or more diffraction peaks at 2θ values selected from 4.85±0.20, 8.14±0.20, 9.02±0.20, 12.58±0.20, 14.20±0.20, 16.85±0.20, 17.31±0.20, 17.66±0.20, 19.28±0.20, 19.92±0.20, 21.36±0.20, 21.72±0.20, 22.38±0.20, 22.75±0.20, 26.34±0.20, 28.85±0.20, and 29.47±0.20.

14. The compound of claim 13 wherein the powder x-ray diffraction pattern comprises two or more diffraction peaks at 2θ values selected from 12.58±0.20, 14.20±0.20, 16.85±0.20, 17.31±0.20, and 17.66±0.20.

15. The compound of claim 12, wherein the compound is characterized by an powder x-ray diffraction pattern in which the peak positions are substantially in accordance with the peak positions of the pattern shown in FIG. 7.

16. A pharmaceutical composition comprising a pharmaceutically acceptable carrier and a compound of claim 1.

17. A method of treating a mammal having a medical condition associated with 5-HT₄ receptor activity, the method comprising administering to the mammal, a therapeutically effective amount of a pharmaceutical composition comprising a pharmaceutically-acceptable carrier and a compound of claim 1.

18. The method of claim 17 wherein the medical condition is selected from the group consisting of irritable bowel syndrome, chronic constipation, functional dyspepsia, delayed gastric emptying, gastroesophageal reflux disease, diabetic gastroparesis, post-operative ileus, intestinal pseudo-obstruction, and drug-induced delayed transit.

19. A method of treating a disorder of reduced motility of the gastrointestinal tract in a mammal, the method comprising administering to the mammal, a therapeutically effective amount of a pharmaceutical composition comprising a pharmaceutically-acceptable carrier and a compound of claim 1.

20. The method of claim 19, wherein the disorder of reduced motility is selected from the group consisting of chronic constipation, constipation-predominant irritable bowel syndrome, diabetic and idiopathic gastroparesis, and functional dyspepsia.

21. A process for preparing a crystalline halide salt of 1-isopropyl-1H-indazole-3-carboxylic acid {(1S,3R,5R)-8-[2-(4-acetylpiperazin-1-yl)ethyl]-8-azabicyclo[3.2.1]oct-3-yl}amide, the process comprising contacting 1-isopropyl-1H-indazole-3-carboxylic acid {(1S,3R,5R)-8-[2-(4-acetylpiperazin-1-yl)ethyl]-8-azabicyclo[3.2.1]oct-3-yl}amide with a halide acid.

22. A process for preparing 1-isopropyl-1H-indazole-3-carboxylic acid {(1S,3R,5R)-8-[2-(4-acetyl-piperazin-1-yl)-ethyl]-8-azabicyclo[3.2.1]oct-3-yl}amide, the process comprising:
(a) reacting (1R,5S)-8-[2-(4-acetylpiperazin-1-yl)ethyl]-8-azabicyclo-[3.2.1]-octan-3-one with an excess of ammonium formate in the presence of a transitional metal catalyst, to provide 1-{4-[2-((1R,3R,5S)-3-amino-8-azabicyclo[3.2.1]oct-8-yl)ethyl]piperazin-1-yl}ethanone; and
(b) reacting 1-{4-[2-((1R,3R,5S)-3-amino-8-azabicyclo[3.2.1]oct-8-yl)ethyl]-piperazin-1-yl}ethanone with 1-isopropyl-1H-indazole-3-carboxylic acid, in the presence of a coupling agent and a base;
to provide 1-isopropyl-1H-indazole-3-carboxylic acid {(1S,3R,5R)-8-[2-(4-acetyl-piperazin-1-yl)-ethyl]-8-azabicyclo[3.2.1]oct-3-yl}amide, or a pharmaceutically-acceptable salt or solvate or stereoisomer thereof.
